(19) United States
(12) Patent Application Publication
(10) Pub. No.: US 2013/0225424 A1
(43) Pub. Date: Aug. 29, 2013

(54) METHODS FOR DETERMINING RESPONSIVENESS TO A DRUG BASED UPON DETERMINATION OF RAS MUTATION AND/OR RAS AMPLIFICATION

(71) Applicant: Sarah S. Bacus, Hinsdale, IL (US)

(72) Inventor: Sarah S. Bacus, Hinsdale, IL (US)

(73) Assignee: Targeted Molecular Diagnostics, LLC, Westmont, IL (US)

(21) Appl. No.: 13/582,146

(22) PCT Filed: Mar. 3, 2011
(86) PCT No.: PCT/US11/27040

§ 371 Date: Feb. 15, 2013

Related U.S. Application Data
(60) Provisional application No. 61/310,030, filed on Mar. 3, 2010.

(51) Int. Cl. C12Q 1/68

(52) U.S. Cl. CPC C12Q 1/6876
USPC 506/7; 435/6.12; 435/6.11

(57) ABSTRACT

The present disclosure provides methods for predicting the sensitivity (e.g., responsiveness) of a cell and/or biological sample obtained from a subject (e.g., a human) to a drug (e.g., a DHFR inhibitor). Such methods may comprise determining the presence or absence of one or more Ras mutations and/or determining the presence or absence of an amplification of the Ras gene in the cell and/or biological sample. The methods may be used to predict the responsiveness of a subject to treatment with a drug.

### FIELD

The present disclosure provides methods for predicting the sensitivity (e.g., responsiveness) of a cell and/or biological sample obtained from a subject to a drug (e.g., a DHFR inhibitor) by determining the presence or absence of one or more Ras mutations and determining the presence or absence of an amplification of the Ras gene in the cell and/or biological sample.

### BACKGROUND

Folate (folic acid) is a vitamin that is essential for the life-sustaining processes of DNA synthesis, replication, and repair. Folate is also important for protein biosynthesis, another process that is central to cell viability. The pteridine compound, methotrexate (MTX), is structurally similar to folate and as a result can bind to the active sites of a number of enzymes that normally use folate as a coenzyme for biosynthesis of purine and pyrimidine nucleotide precursors of DNA and for interconversion of amino acids during protein biosynthesis. Despite its structural similarity to folic acid, methotrexate cannot be used as a cofactor by enzymes that require folate, and instead competes with the folate cofactor for enzyme binding sites, thereby inhibiting protein and DNA biosynthesis and, hence, cell division.

The ability of the folate antagonist methotrexate to inhibit cell division has been exploited in the treatment of a number of diseases and conditions that are characterized by rapid or aberrant cell growth such as cancer and autoimmune disease. As an example, autoimmune diseases are characterized by an inappropriate immune response directed against normal autologous tissues and mediated by rapidly replicating T-cells or B-cells. Autoimmune diseases that have been treated with methotrexate include, without limitation, rheumatoid arthritis and other forms of arthritis, psoriasis, multiple sclerosis, the autoimmune stage of diabetes mellitus (juvenile-onset or Type 1 diabetes), autoimmune uveoretinitis, myasthenia gravis, autoimmune thyroiditis, and systemic lupus erythematosus. A major drawback of methotrexate therapy is inter-patient variability in the clinical response (Weinblatt et al., Arthritis Rheum. 37:1492-1498 (1994); and Walker et al, Arthritis Rheum. 36:329-335 (1993)). Thus, there exists a need for methods that can predict those patients likely to respond to treatment with methotrexate.

### SUMMARY

The present disclosure provides methods for predicting the sensitivity (e.g., clinical responsiveness) of a cell and/or biological sample obtained from a subject (e.g., a human) to a drug (e.g., a DHFR inhibitor). Such methods may comprise determining the presence or absence of one or more Ras mutations (e.g., the number of Ras mutations and/or the level of expression of one or more mutated Ras proteins) in the cell and/or biological sample. The methods may further comprise determining if the cell/biological sample has one or more Ras amplifications (e.g., 3, 4, 5, 6, 7, 8, 9, 10, 11, 12, 13, 14, 15, 16, 17, 18, 19, 20 or more copies of the Ras gene). The methods may be used to predict the responsiveness, including the likelihood of clinical responsiveness, of a subject to treatment with a drug.

The present disclosure provides methods for predicting sensitivity of a test cell to a DHFR inhibitor, by obtaining a test cell; assaying the test cell for one or more Ras mutations; determining if one or more Ras mutations are present or absent in the test cell; and employing the determination of the presence or absence of a Ras mutation in the test cell to predict sensitivity of the test cell to the drug.

In some embodiments, Ras is k-Ras (SEQ ID NO: 1), n-Ras (SEQ ID NO: 2) or h-Ras (SEQ ID NO: 3). In some embodiments, the k-Ras mutations are at one or more of positions 12, 13 or 61. In some embodiments, the k-Ras mutations are selected from the group consisting of: G12A, G12N, G12R, G12C, G125, G12V, G13N and Q61H. In some embodiments, the h-Ras or n-Ras mutations are at one or more of positions 12, 13 or 61.

In some embodiments, the DHFR inhibitor is Methotrexate or Pemetrexed.

In some embodiments, the test cell is obtained from a subject that has a disease or disorder. In some embodiments, the disease or disorder is cancer. In some embodiments, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments, the subject is a cancer patient.

In some embodiments, the test cell is assayed for one or more Ras mutations by analyzing nucleic acid obtained from the test cell. In some embodiments, the test cell is assayed for one or more Ras mutations by analyzing proteins obtained from the test cell.

In some embodiments, test cell is obtained from a tumor biopsy. In some embodiments, the test cell is obtained from an aspirate, blood or serum.

In some embodiments, the test cell is predicted to be sensitive to the DHFR inhibitor where one or more Ras mutations are determined to be present in the test cell. In some embodiments, the test cell is predicted to be sensitive to the DHFR inhibitor where Ras mutations are determined to be absent in the test cell. In some embodiments, the test cell is predicted to be insensitive to the DHFR inhibitor where one or more Ras mutations are determined to be present in the test cell. In some embodiments, the test cell is predicted to be insensitive to the DHFR inhibitor where one or more Ras mutations are determined to be absent in the test cell.

In some embodiments, the step of assaying the test cell for one or more Ras mutations is performed by in situ hybridization (ISH), Northern blot, qRT-PCR or microarray analysis.

The present disclosure also provides methods for selecting subjects for inclusion in a clinical trial for testing the efficacy or safety of a DHFR inhibitor by obtaining a biological sample comprising target cells from the subject; assaying target cells in the biological sample for one or more Ras mutations; determining if one or more Ras mutations are present or absent in the target cells; employing the determination of the presence or absence of a Ras mutation in the target cells to predict sensitivity of the target cells to the DHFR inhibitor; and selecting subjects for inclusion in the clinical that are predicted to be responsive to the DHFR inhibitor.

In some embodiments, subjects are selected for the clinical trial that have one or more Ras mutations present in target cells from their biological sample. In some embodiments, subjects are selected for the clinical trial that have one or more Ras mutations absent in target cells from their biological sample.

The present disclosure also provides methods for predicting responsiveness of a subject with a disease or disorder to treatment with a DHFR inhibitor by obtaining a biological sample from the subject; assaying target cells obtained from the biological sample for one or more Ras mutations; determining if one or more Ras mutations are present or absent in the target cells; and employing the determination of the presence or absence of a Ras mutation in the target cells obtained from the biological sample to predict responsiveness of the subject to the DHFR inhibitor.

In some embodiments, the subject is predicted to be responsive to the DHFR inhibitor where one or more Ras mutations are present in the target cells. In some embodiments, the subject is predicted to be responsive to the DHFR inhibitor where one or more Ras mutations are absent in the target cells. In some embodiments, the subject is predicted to be non-responsive to the DHFR inhibitor where one or more Ras mutations are present in the target cells. In some embodiments, the subject is predicted to be non-responsive to the DHFR inhibitor where one or more Ras mutations are absent in the target.

The present disclosure also provides methods for treating a subject with a disease or disorder by obtaining a biological sample from a subject; assaying target cells obtained from the biological sample for one or more Ras mutations; determining if one or more Ras mutations are present or absent in the target cells; employing the determination of the presence or absence of a Ras mutation in the target cells obtained from the biological sample to predict responsiveness of the subject to a DHFR inhibitor; and administering to the subject a therapeutically effective amount of the DHFR inhibitor where the subject is predicted to be responsive to the DHFR inhibitor.

In some embodiments, the subject is predicted to be responsive to the DHFR inhibitor where one or more Ras mutations are present in the target cells. In some embodiments, the subject is predicted to be responsive to the drug where one or more Ras mutations are absent in the target cells.

The present disclosure also has methods for predicting sensitivity of a test cell to an DHFR inhibitor by obtaining a test cell; assaying the test cell for one or more Ras mutations; determining if the test cell has one or more Ras mutations; and employing the determination of the presence of absence of a Ras mutation in the test cell to predict sensitivity of the test cell to the DHFR inhibitor, wherein the presence of a Ras mutation predicts that the test cell will be sensitive to the DHFR inhibitor, the absence of a Ras mutation predicts that the test cell will be sensitive to the DHFR inhibitor, the presence of a Ras mutation predicts that the test cell will be insensitive to the DHFR inhibitor, or the absence of a Ras mutation predicts that the test cell will be insensitive to the DHFR inhibitor.

The present disclosure provides methods for predicting sensitivity of a test cell to a drug by obtaining a test cell; assaying the test cell for one or more Ras mutations; assaying the test cell for amplification of a Ras gene; determining if one or more Ras mutations are present or absent in the test cell and determining if an amplification of the Ras gene is present or absent in the test cell; and employing the determination of the presence or absence of a Ras mutation in the test cell and the presence or absence of an amplification of Ras in the test cell to predict sensitivity of the test cell to the drug.

In some embodiments, Ras is k-Ras (SEQ ID NO: 1), n-Ras (SEQ ID NO: 2) or h-Ras (SEQ ID NO: 3). In some embodiments, the k-Ras mutations are at one or more of positions 12, 13 or 61. In some embodiments, the k-Ras mutations are selected from the group consisting of: G12A, G12N, G12R, G12C, G125, G12V, G13N and Q61H. In some embodiments, the h-Ras or n-Ras mutations are at one or more of positions 12, 13 or 61.

In some embodiments the Ras amplification is one or more of an amplification of k-Ras (SEQ ID NO: 4), n-Ras (SEQ ID NO: 5) or h-Ras (SEQ ID NO: 6).

In some embodiments, the drug is a chemotherapeutic agent. In some embodiments, the drug is an EGFR targeted therapy. In some embodiments, the drug is an antifolate such as a dihydrofolate reductase (DHFR) inhibitor. In some embodiments, the DHFR inhibitor is Methotrexate or Pemetrexed.

In some embodiments, the EGFR targeted therapy is a tyrosine kinase inhibitor that targets HER1 (EGFR), HER2/neu, HER3, or any combination thereof. In some embodiments, the tyrosine kinase inhibitor is an antibody. In some embodiments, the antibody is a monoclonal antibody. In some embodiments, the monoclonal antibody is cetuximab (Erbitux), panitumumab, zalutumumab, nimotuzumab or matuzumab. In some embodiments, the tyrosine kinase inhibitor is a small molecule inhibitor. In some embodiments, the small molecule inhibitor is gefitinib, erlotinib or lapatinib.

In some embodiments, the test cell is obtained from a subject that has a disease or disorder. In some embodiments, the subject is a cancer patient.

In some embodiments, the disease or disorder is cancer. In some embodiments, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments, the test cell is assayed for one or more Ras mutations and an amplification of Ras by analyzing nucleic acid obtained from the test cell. In some embodiments, the test cell is assayed for one or more Ras mutations by analyzing proteins obtained from the test cell.

In some embodiments, the test cell is obtained from a tumor biopsy. In some embodiments, the test cell is obtained from an aspirate, blood or serum.

In some embodiments, the test cell is predicted to be sensitive to the drug where one or more Ras mutations are determined to be present in the test cell and an amplification of Ras is determined to be present in the test cell.

In some embodiments, the test cell is predicted to be sensitive to the drug where one or more Ras mutations are determined to be present in the test cell and amplification of Ras is determined to be absent in the test cell.

In some embodiments, the test cell is predicted to be sensitive to the drug where Ras mutations are determined to be absent in the test cell and an amplification of Ras is determined to be present in the test cell.

In some embodiments, the test cell is predicted to be sensitive to the drug where Ras mutations are determined to be absent in the test cell and amplification of Ras is determined to be absent in the test cell.

In some embodiments, the test cell is predicted to be insensitive to the drug where one or more Ras mutations are determined to be present in the test cell and an amplification of Ras is determined to be present in the test cell.

In some embodiments, the test cell is predicted to be insensitive to the drug where one or more Ras mutations are determined to be present in the test cell and amplification of Ras is determined to be absent in the test cell.

In some embodiments, the test cell is predicted to be insensitive to the drug where Ras mutations are determined to be absent in the test cell and an amplification of Ras is determined to be present in the test cell.

In some embodiments, the test cell is predicted to be insensitive to the drug where Ras mutations are determined to be absent in the test cell and amplification of Ras is determined to be absent in the test cell.

In some embodiments, the step of assaying the test cell for one or more Ras mutations and amplification of Ras is performed by in situ hybridization (ISH), Northern blot, qRT-PCR or microarray analysis.

The present disclosure also provides methods for selecting subjects for inclusion in a clinical trial including, a clinical trial for testing the efficacy or safety of a drug, by obtaining a biological sample comprising target cells from the subject; assaying target cells in the biological sample for one or more Ras mutations; assaying target cells in the biological sample for an amplification Ras; determining if one or more Ras mutations are present or absent in the target cells and determining if an amplification of the Ras gene is present or absent in the target cells; employing the determination of the presence or absence of a Ras mutation in the target cells and the presence or absence of an amplification of Ras in the target cells to predict sensitivity of the target cells to the drug; and selecting subjects for inclusion in the clinical that are predicted to be responsive to the drug.

The present disclosure provides methods for selecting subjects for inclusion in a clinical trial for testing the efficacy or safety of a drug by obtaining a biological sample comprising target cells from the subject; determining if the cells have one or more Ras mutations; determining if the cells have a Ras amplification; and selecting subjects for inclusion in the clinical with the drug based upon the determination of whether the target cells have one or more Ras mutations and a Ras amplification.

In an embodiment, the subjects that have one or more Ras mutations and a Ras amplification are selected for inclusion in the clinical trial. In another embodiment, the subjects that have one or more Ras mutations and do not have a Ras amplification are selected for inclusion in the clinical trial. In yet another embodiment, the subjects that do not have one or more Ras mutations and have a Ras amplification are selected for inclusion in the clinical trial. In another embodiment, the subjects that do not have one or more Ras mutations and do not have a Ras amplification are selected for inclusion in the clinical trial.

In some embodiments, subjects are selected for the clinical trial that have one or more Ras mutations present in target cells from their biological sample and that have an amplification of Ras present in target cells from their biological sample.

In some embodiments, subjects are selected for the clinical trial that have one or more Ras mutations absent in target cells from their biological sample and that have an amplification of Ras present in target cells from their biological sample.

In some embodiments, subjects are selected for the clinical trial that have one or more Ras mutations present in target cells from their biological sample and that have an amplification of Ras absent in target cells from their biological sample.

In some embodiments, subjects are selected for the clinical trial that have one or more Ras mutations absent in target cells from their biological sample and that have an amplification of Ras absent in target cells from their biological sample.

In some embodiments, Ras is k-Ras (SEQ ID NO: 1), n-Ras (SEQ ID NO: 2) or h-Ras (SEQ ID NO: 3). In some embodiments, the k-Ras mutations are at one or more of positions 12, 13 or 61. In some embodiments, the k-Ras mutations are selected from the group consisting of: G12A, G12N, G12R, G12C, G125, G12V, G13N and Q61H. In some embodiments, the h-Ras or n-Ras mutations are at one or more of positions 12, 13 or 61.

In some embodiments the Ras amplification is one or more of an amplification of k-Ras (SEQ ID NO: 4), n-Ras (SEQ ID NO: 5) or h-Ras (SEQ ID NO: 6).

In some embodiments, the drug is a chemotherapeutic agent. In some embodiments, the drug is an EGFR targeted therapy. In some embodiments, the drug is an antifolate such as a dihydrofolate reductase (DHFR) inhibitor. In some embodiments, the DHFR inhibitor is Methotrexate or Pemetrexed.

In some embodiments, the EGFR targeted therapy is a tyrosine kinase inhibitor that targets HER1 (EGFR), HER2/neu, HER3, or any combination thereof. In some embodiments, the tyrosine kinase inhibitor is an antibody. In some embodiments, the antibody is a monoclonal antibody. In some embodiments, the monoclonal antibody is cetuximab (Erbitux), panitumumab, zalutumumab, nimotuzumab or matuzumab. In some embodiments, the tyrosine kinase inhibitor is a small molecule inhibitor. In some embodiments, the small molecule inhibitor is gefitinib, erlotinib or lapatinib.

In some embodiments, the biological sample is obtained from a subject that has a disease or disorder. In some embodiments, the subject is a cancer patient.

In some embodiments, the disease or disorder is cancer. In some embodiments, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments, the biological sample (e.g., one or more cells in the biological sample) is assayed for one or more Ras mutations and an amplification of Ras by analyzing nucleic acid obtained from the test cell. In some embodiments, the biological sample (e.g., one or more cells in the biological sample) is assayed for one or more Ras mutations by analyzing proteins obtained from the test cell.

In some embodiments, the biological sample is obtained from a tumor biopsy. In some embodiments, the biological sample is obtained from an aspirate, blood or serum.

In some embodiments, the step of assaying target cells in the biological sample for one or more Ras mutations and amplification of Ras is performed by in situ hybridization (ISH), Northern blot, qRT-PCR or microarray analysis.

The present disclosure also provides methods for predicting responsiveness of a subject with a disease or disorder to treatment with a drug by obtaining a biological sample from the subject; assaying target cells obtained from the biological sample for one or more Ras mutations; assaying target cells obtained from the biological sample for a Ras amplification; determining if one or more Ras mutations are present or absent in the target cells and determining if an amplification of the Ras gene is present or absent in the target cells; and employing the determination of the presence or absence of a Ras mutation and the presence or absence of an amplification of Ras in the target cells obtained from the biological sample to predict responsiveness of the subject to the drug.

In some embodiments, the subject is predicted to be responsive to the drug where one or more Ras mutations are present in the target cells and an amplification of Ras is present in the target cells.

In some embodiments, the subject is predicted to be responsive to the drug where one or more Ras mutations are absent in the target cells and an amplification of Ras is present in the target cells.

In some embodiments, the subject is predicted to be responsive to the drug where one or more Ras mutations are present in the target cells and an amplification of Ras is absent in the target cells.

In some embodiments, the subject is predicted to be responsive to the drug where one or more Ras mutations are absent in the target cells and an amplification of Ras is absent in the target cells.

In some embodiments, the subject is predicted to be non-responsive to the drug where one or more Ras mutations are present in the target cells and an amplification of Ras is present in the target cells.

In some embodiments, the subject is predicted to be non-responsive to the drug where one or more Ras mutations are absent in the target cells and an amplification of Ras is present in the target cells.

In some embodiments, the subject is predicted to be non-responsive to the drug where one or more Ras mutations are present in the target cells and an amplification of Ras is absent in the target cells.

In some embodiments, the subject is predicted to be non-responsive to the drug where one or more Ras mutations are absent in the target cells and an amplification of Ras is absent in the target cells.

In some embodiments, Ras is k-Ras (SEQ ID NO: 1), n-Ras (SEQ ID NO: 2) or h-Ras (SEQ ID NO: 3). In some embodiments, the k-Ras mutations are at one or more of positions 12, 13 or 61. In some embodiments, the k-Ras mutations are selected from the group consisting of: G12A, G12N, G12R, G12C, G125, G12V, G13N and Q61H. In some embodiments, the h-Ras or n-Ras mutations are at one or more of positions 12, 13 or 61.

In some embodiments the Ras amplification is one or more of an amplification of k-Ras (SEQ ID NO: 4), n-Ras (SEQ ID NO: 5) or h-Ras (SEQ ID NO: 6).

In some embodiments, the drug is a chemotherapeutic agent. In some embodiments, the drug is an EGFR targeted therapy. In some embodiments, the drug is an antifolate such as a dihydrofolate reductase (DHFR) inhibitor. In some embodiments, the DHFR inhibitor is Methotrexate or Pemetrexed.

In some embodiments, the EGFR targeted therapy is a tyrosine kinase inhibitor that targets HER1 (EGFR), HER2/neu, HER3, or any combination thereof. In some embodiments, the tyrosine kinase inhibitor is an antibody. In some embodiments, the antibody is a monoclonal antibody. In some embodiments, the monoclonal antibody is cetuximab (Erbitux), panitumumab, zalutumumab, nimotuzumab or matuzumab. In some embodiments, the tyrosine kinase inhibitor is a small molecule inhibitor. In some embodiments, the small molecule inhibitor is gefitinib, erlotinib or lapatinib.

In some embodiments, the biological sample is obtained from a subject that has a disease or disorder. In some embodiments, the subject is a cancer patient.

In some embodiments, the disease or disorder is cancer. In some embodiments, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments, the biological sample (e.g., one or more cells in the biological sample) is assayed for one or more Ras mutations and an amplification of Ras by analyzing nucleic acid obtained from the test cell. In some embodiments, the biological sample (e.g., one or more cells in the biological sample) is assayed for one or more Ras mutations by analyzing proteins obtained from the test cell.

In some embodiments, the biological sample is obtained from a tumor biopsy. In some embodiments, the biological sample is obtained from an aspirate, blood or serum.

In some embodiments, the step of assaying target cells in the biological sample for one or more Ras mutations and amplification of Ras is performed by in situ hybridization (ISH), Northern blot, qRT-PCR or microarray analysis.

The present disclosure also provides methods for treating a subject with a disease or disorder by obtaining a biological sample from a subject; assaying target cells obtained from the biological sample for one or more Ras mutations; assaying target cells obtained from the biological sample for a Ras amplification; determining if one or more Ras mutations are present or absent in the target cells and determining if an amplification of the Ras gene is present or absent in the target cells; employing the determination of the presence or absence of a Ras mutation and the presence or absence of an amplification of Ras in the target cells obtained from the biological sample to predict responsiveness of the subject to a drug; and administering to the subject a therapeutically effective amount of the drug where the subject is predicted to be responsive to the drug.

In some embodiments, the subject is predicted to be responsive to the drug where one or more Ras mutations are present in the target cells and an amplification of Ras is present in the target cells.

In some embodiments, the subject is predicted to be responsive to the drug where one or more Ras mutations are absent in the target cells and an amplification of Ras is present in the target cells.

In some embodiments, the subject is predicted to be responsive to the drug where one or more Ras mutations are present in the target cells and an amplification of Ras is absent in the target cells.

In some embodiments, the subject is predicted to be responsive to the drug where one or more Ras mutations are absent in the target cells and an amplification of Ras is absent in the target cells.

In some embodiments, Ras is k-Ras (SEQ ID NO: 1), n-Ras (SEQ ID NO: 2) or h-Ras (SEQ ID NO: 3). In some embodiments, the k-Ras mutations are at one or more of positions 12, 13 or 61. In some embodiments, the k-Ras mutations are selected from the group consisting of: G12A, G12N, G12R, G12C, G125, G12V, G13N and Q61H. In some embodiments, the h-Ras or n-Ras mutations are at one or more of positions 12, 13 or 61.

In some embodiments the Ras amplification is one or more of an amplification of k-Ras (SEQ ID NO: 4), n-Ras (SEQ ID NO: 5) or h-Ras (SEQ ID NO: 6).

In some embodiments, the drug is a chemotherapeutic agent. In some embodiments, the drug is an EGFR targeted therapy. In some embodiments, the drug is an antifolate such as a dihydrofolate reductase (DHFR) inhibitor. In some embodiments, the DHFR inhibitor is Methotrexate or Pemetrexed.

In some embodiments, the EGFR targeted therapy is a tyrosine kinase inhibitor that targets HER1 (EGFR), HER2/neu, HER3, or any combination thereof. In some embodiments, the tyrosine kinase inhibitor is an antibody. In some embodiments, the antibody is a monoclonal antibody. In some embodiments, the monoclonal antibody is cetuximab (Erbitux), panitumumab, zalutumumab, nimotuzumab or matuzumab. In some embodiments, the tyrosine kinase inhibitor is a small molecule inhibitor. In some embodiments, the small molecule inhibitor is gefitinib, erlotinib or lapatinib.

In some embodiments, the biological sample is obtained from a subject that has a disease or disorder. In some embodiments, the subject is a cancer patient.

In some embodiments, the disease or disorder is cancer. In some embodiments, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments, the biological sample (e.g., one or more cells in the biological sample) is assayed for one or more Ras mutations and an amplification of Ras by analyzing nucleic acid obtained from the test cell. In some embodiments, the biological sample (e.g., one or more cells in the biological sample) is assayed for one or more Ras mutations by analyzing proteins obtained from the test cell.

In some embodiments, the biological sample is obtained from a tumor biopsy. In some embodiments, the biological sample is obtained from an aspirate, blood or serum.

In some embodiments, the step of assaying target cells in the biological sample for one or more Ras mutations and amplification of Ras is performed by in situ hybridization (ISH), Northern blot, qRT-PCR or microarray analysis.

The present disclosure also provides methods for predicting sensitivity of a test cell to a DHFR inhibitor by obtaining a test cell; assaying the test cell for one or more Ras mutations; assaying the test cell for amplification of a Ras gene; determining if the test cell has one or more Ras mutations and an amplification of the Ras gene; and employing the determination of the presence of absence of a Ras mutation and amplification of Ras in the test cell to predict sensitivity of the test cell to the drug, wherein the presence of a Ras mutation and the presence of an amplification of Ras predicts that the test cell will be insensitive to the DHFR inhibitor, the presence of a Ras mutation and the absence of a Ras amplification predicts that the test cell will be insensitive to the DHFR inhibitor, the absence of a Ras mutation and the presence of an amplification of Ras predicts that the test cell will be sensitive to the DHFR inhibitor or the absence of a Ras mutation and the absence of an amplification of Ras predicts that the test cell will be insensitive to the DHFR inhibitor.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows the G150 (μL) for K-Ras mutant versus K-Ras wild type NCI-60 NSCLC cell lines treated with antifolates such as Methotrexate, Trimetrexate, soluble bakers antifol, or %-fluorouracil.

FIG. 2 shows a growth curve of K-Ras mutant, K-Ras mutant and K-Ras amplified, and K-Ras wild type cells treated with Methotrexate.

FIG. 3 shows an RT-PCR analysis of gene expression of K-Ras, E2F1 and DHFR in A549 cells treated with Methotrexate.

FIG. 4 shows the in vivo responsiveness of K-Ras mutant tumors to Methotrexate.

### DETAILED DESCRIPTION

Several recent clinical studies have shown that the presence of a Ras mutation, such as K-Ras, is a significant predictor of non-responsiveness (e.g., resistance) to treatment with a drug such as a receptor tyrosine kinase inhibitor including, for example, an EGFR inhibitor (e.g. Erlotinib, Gefitinib). However, the inventors of the instant disclosure have unexpectedly demonstrated that cells which harbor a Ras mutation are likely to respond differently than cells which do not harbor a Ras mutation to a dihydrofolate reductase (DHFR) inhibitor such as Methotrexate or Pemetrexed (ALTIMA™). Thus, contrary to conventional wisdom, mutation of Ras is not a sole predictor of resistance to a targeted therapy or a chemotherapy. Instead, the inventors of the instant disclosure have unexpectedly demonstrated that cells which harbor a Ras mutation (e.g., a K-Ras mutation) are exquisitely sensitive to a drug including, a dihydrofolate reductase (DHFR) inhibitor such as Methotrexate or Pemetrexed (ALTIMA™) as compared to a cell with wild type Ras. Accordingly, the present methods and materials may be used to select subjects for inclusion/exclusion in a clinical trial, predict the responsiveness of a subject to a drug (e.g., a DHFR inhibitor) and/or select a drug that will elicit a response in a subject. Further, a drug predicted to elicit a response in a subject may be used to treat a disease or disorder including, for example, cancer or an autoimmune disease characterized by aberrant cell proliferation.

The present disclosure provides methods for predicting sensitivity of a test cell to a DHFR inhibitor, by obtaining a test cell; assaying the test cell for one or more Ras mutations (e.g., one or more mutations in k-Ras (SEQ ID NO: 1), n-Ras (SEQ ID NO: 2) or h-Ras (SEQ ID NO: 3); determining if one or more Ras mutations are present or absent (e.g., Ras wild type) in the test cell; and employing the determination of the presence or absence of a Ras mutation in the test cell to predict sensitivity of the test cell to the DHFR inhibitor. Optionally, the test cell may be assayed for a Ras amplification and the determination of the presence or absence of a Ras amplification in the test cell may be used with the determination of the absence or presence of a Ras mutation to predict sensitivity of the test cell to the DHFR inhibitor.

The present disclosure also provides methods for selecting subjects for inclusion in a clinical trial for testing the efficacy or safety of a DHFR inhibitor by obtaining a biological sample comprising target cells from the subject; assaying target cells in the biological sample for one or more Ras mutations (e.g., one or more mutations in k-Ras (SEQ ID NO: 1), n-Ras (SEQ ID NO: 2) or h-Ras (SEQ ID NO: 3); determining if one or more Ras mutations are present or absent (e.g., Ras wild type) in the target cells; employing the determination of the presence or absence of a Ras mutation in the target cells to predict sensitivity of the target cells to the DHFR inhibitor; and selecting subjects for inclusion in the clinical that are predicted to be responsive to the DHFR inhibitor. Optionally, the target cells may be assayed for a Ras amplification and the determination of the presence or absence of a Ras amplification in the target cells may be used with the determination of the absence or presence of a Ras mutation to predict sensitivity of the target cells to the DHFR inhibitor.

The present disclosure also provides methods for predicting responsiveness of a subject with a disease or disorder to treatment with a DHFR inhibitor by obtaining a biological sample from the subject; assaying target cells obtained from the biological sample for one or more Ras mutations (e.g., one or more mutations in k-Ras (SEQ ID NO: 1), n-Ras (SEQ ID NO: 2) or h-Ras (SEQ ID NO: 3); determining if one or more Ras mutations are present or absent (e.g., Ras wild type) in the target cells; and employing the determination of the presence or absence of a Ras mutation in the target cells obtained from the biological sample to predict responsiveness of the subject to the DHFR inhibitor. Optionally, the target cells may be assayed for a Ras amplification and the determination of the presence or absence of a Ras amplification in the target cells may be used with the determination of the absence or presence of a Ras mutation to predict sensitivity of the target cells to the DHFR inhibitor. A subject predicted to be responsive with a DHFR inhibitor may be administered the DHFR inhibitor with or without an additional therapy including, for example, an EGFR targeted therapy.

The present disclosure also provides methods for treating a subject with a disease or disorder by obtaining a biological sample from a subject; assaying target cells obtained from the biological sample for one or more Ras mutations (e.g., one or more mutations in k-Ras (SEQ ID NO: 1), n-Ras (SEQ ID NO: 2) or h-Ras (SEQ ID NO: 3); determining if one or more Ras mutations are present or absent (e.g., Ras wild type) in the target cells; employing the determination of the presence or absence of a Ras mutation in the target cells obtained from the biological sample to predict responsiveness of the subject to a DHFR inhibitor; and administering to the subject a therapeutically effective amount of the DHFR inhibitor where the subject is predicted to be responsive to the DHFR inhibitor. Optionally, the target cells may be assayed for a Ras amplification and the determination of the presence or absence of a Ras amplification in the target cells may be used with the determination of the absence or presence of a Ras mutation to predict sensitivity of the target cells to the DHFR inhibitor. A subject predicted to be responsive with a DHFR inhibitor may be administered the DHFR inhibitor with or without an additional therapy including, for example, an EGFR targeted therapy.

The present disclosure also has methods for predicting sensitivity of a test cell to an DHFR inhibitor by obtaining a test cell; assaying the test cell for one or more Ras mutations; determining if the test cell has one or more Ras mutations (e.g., one or more mutations in k-Ras (SEQ ID NO: 1), n-Ras (SEQ ID NO: 2) or h-Ras (SEQ ID NO: 3); and employing the determination of the presence of absence of a Ras mutation in the test cell to predict sensitivity of the test cell to the DHFR inhibitor, wherein the presence of a Ras mutation predicts that the test cell will be sensitive to the DHFR inhibitor, the absence of a Ras mutation predicts that the test cell will be sensitive to the DHFR inhibitor, the presence of a Ras mutation predicts that the test cell will be insensitive to the DHFR inhibitor, or the absence of a Ras mutation predicts that the test cell will be insensitive to the DHFR inhibitor. Optionally, the test cell may be assayed for a Ras amplification and the determination of the presence or absence of a Ras amplification in the test cell may be used with the determination of the absence or presence of a Ras mutation to predict sensitivity of the target cells to the DHFR inhibitor.

The present disclosure provides methods for predicting sensitivity of a test cell (e.g., a cell obtained from a cancer patient) to a drug (e.g., an antifolate such as a dihydrofolate reductase (DHFR); or an EGFR inhibitor) by obtaining a test cell; assaying the test cell for one or more Ras mutations (e.g., one or more mutations in k-Ras (SEQ ID NO: 1), n-Ras (SEQ ID NO: 2) or h-Ras (SEQ ID NO: 3); assaying the test cell for amplification of a Ras gene (e.g., an amplification of one or more of k-Ras (SEQ ID NO: 4), n-Ras (SEQ ID NO: 5) or h-Ras (SEQ ID NO: 6)); determining if one or more Ras mutations are present or absent in the test cell and determining if an amplification of the Ras gene is present or absent in the test cell; and employing the determination of the presence or absence of a Ras mutation (e.g., Ras wild type) in the test cell and the presence or absence of an amplification of Ras in the test cell to predict sensitivity of the test cell to the drug. In some embodiments, the test cell is predicted to be sensitive to the drug where one or more Ras mutations are determined to be present in the test cell and an amplification of Ras is determined to be present in the test cell. In some embodiments, the test cell is predicted to be sensitive to the drug where one or more Ras mutations are determined to be present in the test cell and amplification of Ras is determined to be absent in the test cell. In some embodiments, the test cell is predicted to be sensitive to the drug where Ras mutations are determined to be absent (e.g., Ras wild type) in the test cell and an amplification of Ras is determined to be present in the test cell. In some embodiments, the test cell is predicted to be sensitive to the drug where Ras mutations are determined to be absent in the test cell (e.g., Ras wild type) and amplification of Ras is determined to be absent in the test cell. In some embodiments, the test cell is predicted to be insensitive to the drug where one or more Ras mutations are determined to be present in the test cell and an amplification of Ras is determined to be present in the test cell. In some embodiments, the test cell is predicted to be insensitive to the drug where one or more Ras mutations are determined to be present in the test cell and amplification of Ras is determined to be absent in the test cell. In some embodiments, the test cell is predicted to be insensitive to the drug where Ras mutations are determined to be absent (e.g., Ras wild type) in the test cell and an amplification of Ras is determined to be present in the test cell. In some embodiments, the test cell is predicted to be insensitive to the drug where Ras mutations are determined to be absent (e.g., Ras wild type) in the test cell and amplification of Ras is determined to be absent in the test cell.

The present disclosure also provides methods for selecting subjects for inclusion in a clinical trial for testing the efficacy or safety of a drug (e.g., an antifolate such as a dihydrofolate reductase (DHFR); or an EGFR inhibitor) by obtaining a biological sample (e.g., a biological sample obtained from a cancer patient) comprising target cells from the subject; assaying target cells in the biological sample for one or more Ras mutations (e.g., one or more mutations in k-Ras (SEQ ID NO: 1), n-Ras (SEQ ID NO: 2) or h-Ras (SEQ ID NO: 3); assaying target cells in the biological sample for an amplification of Ras (e.g., an amplification of one or more of k-Ras (SEQ ID NO: 4), n-Ras (SEQ ID NO: 5) or h-Ras (SEQ ID NO: 6)); determining if one or more Ras mutations are present or absent in the target cells and determining if an amplification of the Ras gene is present or absent in the target cells; employing the determination of the presence or absence of a Ras mutation in the target cells and the presence or absence of an amplification of Ras in the target cells to predict sensitivity of the target cells to the drug; and selecting subjects for inclusion in the clinical that are predicted to be responsive to the drug. In some embodiments, subjects are selected for the clinical trial that have one or more Ras mutations present in target cells from their biological sample and that have an amplification of Ras present in target cells from their biological sample. In some embodiments, subjects are selected for the clinical trial that have one or more Ras mutations absent (e.g., Ras wild type) in target cells from their biological sample and that have an amplification of Ras present in target cells from their biological sample. In some embodiments, subjects are selected for the clinical trial that have one or more Ras mutations present in target cells from their biological sample and that have an amplification of Ras absent in target cells from their biological sample. In some embodiments, subjects are selected for the clinical trial that have one or more Ras mutations absent (e.g., Ras wild type) in target cells from their biological sample and that have an amplification of Ras absent in target cells from their biological sample.

The present disclosure also provides methods for predicting responsiveness of a subject with a disease or disorder to treatment with a drug (e.g., an antifolate such as a dihydrofolate reductase (DHFR); or an EGFR inhibitor) by obtaining a biological sample (e.g., a biological sample obtained from a cancer patient) from the subject; assaying target cells obtained from the biological sample for one or more Ras mutations (e.g., one or more mutations in k-Ras (SEQ ID NO: 1), n-Ras (SEQ ID NO: 2) or h-Ras (SEQ ID NO: 3); assaying target cells obtained from the biological sample for a Ras amplification (e.g., an amplification of one or more of k-Ras (SEQ ID NO: 4), n-Ras (SEQ ID NO: 5) or h-Ras (SEQ ID NO: 6)); determining if one or more Ras mutations are present or absent in the target cells and determining if an amplification of the Ras gene is present or absent in the target cells; and employing the determination of the presence or absence of a Ras mutation and the presence or absence of an amplification of Ras in the target cells obtained from the biological sample to predict responsiveness of the subject to the drug. In some embodiments, the subject is predicted to be responsive to the drug where one or more Ras mutations are present in the target cells and an amplification of Ras is present in the target cells. In some embodiments, the subject is predicted to be responsive to the drug where one or more Ras mutations are absent (e.g., Ras wild type) in the target cells and an amplification of Ras is present in the target cells. In some embodiments, the subject is predicted to be responsive to the drug where one or more Ras mutations are present in the target cells and an amplification of Ras is absent in the target cells. In some embodiments, the subject is predicted to be responsive to the drug where one or more Ras mutations are absent (e.g., Ras wild type) in the target cells and an amplification of Ras is absent in the target cells. In some embodiments, the subject is predicted to be non-responsive to the drug where one or more Ras mutations are present in the target cells and an amplification of Ras is present in the target cells. In some embodiments, the subject is predicted to be non-responsive to the drug where one or more Ras mutations are absent (e.g., Ras wild type) in the target cells and an amplification of Ras is present in the target cells. In some embodiments, the subject is predicted to be non-responsive to the drug where one or more Ras mutations are present in the target cells and an amplification of Ras is absent in the target cells. In some embodiments, the subject is predicted to be non-responsive to the drug where one or more Ras mutations are absent (e.g., Ras wild type) in the target cells and an amplification of Ras is absent in the target cells.

The present disclosure also provides methods for treating a subject with a disease or disorder by obtaining a biological sample (e.g., a biological sample obtained from a cancer patient) from a subject; assaying target cells obtained from the biological sample for one or more Ras mutations (e.g., one or more mutations in k-Ras (SEQ ID NO: 1), n-Ras (SEQ ID NO: 2) or h-Ras (SEQ ID NO: 3); assaying target cells obtained from the biological sample for a Ras amplification (e.g., an amplification of one or more of k-Ras (SEQ ID NO: 4), n-Ras (SEQ ID NO: 5) or h-Ras (SEQ ID NO: 6)); determining if one or more Ras mutations are present or absent in the target cells and determining if an amplification of the Ras gene is present or absent in the target cells; employing the determination of the presence or absence of a Ras mutation and the presence or absence of an amplification of Ras in the target cells obtained from the biological sample to predict responsiveness of the subject to a drug (e.g., an antifolate such as a dihydrofolate reductase (DHFR); or an EGFR inhibitor); and administering to the subject a therapeutically effective amount of the drug where the subject is predicted to be responsive to the drug. In some embodiments, the subject is predicted to be responsive to the drug where one or more Ras mutations are present in the target cells and an amplification of Ras is present in the target cells. In some embodiments, the subject is predicted to be responsive to the drug where one or more Ras mutations are absent (e.g., Ras wild type) in the target cells and an amplification of Ras is present in the target cells. In some embodiments, the subject is predicted to be responsive to the drug where one or more Ras mutations are present in the target cells and an amplification of Ras is absent in the target cells. In some embodiments, the subject is predicted to be responsive to the drug where one or more Ras mutations are absent (e.g., Ras wild type) in the target cells and an amplification of Ras is absent in the target cells.

The present disclosure also provides methods for predicting sensitivity of a test cell to a DHFR inhibitor by obtaining a test cell (e.g., a cell obtained from a cancer patient); assaying the test cell for one or more Ras mutations (e.g., one or more mutations in k-Ras (SEQ ID NO: 1), n-Ras (SEQ ID NO: 2) or h-Ras (SEQ ID NO: 3); assaying the test cell for amplification of a Ras gene (e.g., an amplification of one or more of k-Ras (SEQ ID NO: 4), n-Ras (SEQ ID NO: 5) or h-Ras (SEQ ID NO: 6)); determining if the test cell has one or more Ras mutations and an amplification of the Ras gene; and employing the determination of the presence of absence of a Ras mutation and amplification of Ras in the test cell to predict sensitivity of the test cell to the drug, wherein the presence of a Ras mutation and the presence of an amplification of Ras predicts that the test cell will be insensitive to the DHFR inhibitor, the presence of a Ras mutation and the absence of a Ras amplification predicts that the test cell will be insensitive to the DHFR inhibitor, the absence of a Ras mutation and the presence of an amplification of Ras predicts that the test cell will be sensitive to the DHFR inhibitor or the absence of a Ras mutation and the absence of an amplification of Ras predicts that the test cell will be insensitive to the DHFR inhibitor.

The present disclosure also provides methods for modulating the responsiveness of a subject to an EGFR targeted therapy including, for example, a DHFR inhibitor such as Methotrexate by obtaining a biological sample comprising target cells from the subject, determining if the cells have one or more Ras mutations; determining if the cells have a Ras amplification, and where it is determined that the subject has a Ras mutation and does not have a Ras amplification; administering to the subject one or more agents that increase expression of Ras (e.g., K-Ras).

Target cells may include, for example, cells to be treated (e.g., killed) by a drug. In some embodiments, target cells may include cancer cells.

In some embodiments, Ras mutation (e.g., mutated Ras) and/or Ras amplification may be detected in formalin-fixed paraffin-embedded (FFPE) tissue samples obtained from a subject.

A cell or biological sample may be considered responsive/sensitive to a drug if the dug induces apoptosis, decreases cell proliferation of the cell and/or biological sample. Responsiveness of a cell or biological sample to a chemotherapeutic agent may also be measured as a reduction in size of the cell or biological sample. In some embodiments, the cell and/or biological sample may be considered responsive/sensitive to a drug where there is a greater than 50%, 60%, 70%, 80%, 90% or 95% likelihood that the cell and/or biological sample will be responsive/sensitive to the drug. In some embodiments, a cell or biological sample may be considered responsive/sensitive to a drug if the dug induces apoptosis, decreases cell proliferation of the cell and/or biological sample as compared to a control cell/control biological sample. Responsiveness of a cell or biological sample to a chemotherapeutic agent may also be measured as a reduction in size of the cell or biological sample as compared to the control cell or control biological sample. In some embodiments, the cell and/or biological sample may be considered responsive/sensitive to a drug where there is a greater than 50%, 60%, 70%, 80%, 90% or 95% likelihood that the cell and/or biological sample will be responsive/sensitive to the drug.

A subject including, for example, a human patient, may be considered responsive/sensitive to a drug if the dug induces apoptosis, decreases cell proliferation, or induces an immune response against a cell and/or biological sample obtained from the subject or patient. Responsiveness of the subject to a chemotherapeutic agent may also be measured as a reduction in size of the cell or biological sample.

A test cell may include a tumor cell. For examination of a long-term treatment effect, or effectiveness for individual patients, namely, tailor made medicine, it is possible to culture a cancer cell that can be obtained from a tumor of patient and use the cancer cell as a test cell.

In some embodiments, patients with a disease or disorder such as cancer or an autoimmune disease that are predicted to be responsive to a drug, including a chemotherapy such as Methotrexate, may be treated with an effective amount of the drug to treat the disease or disorder.

In some embodiments, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The treating or treatment of a disease or disorder may include treating or the treatment of cancer.

The term “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as a compound of Formula (A) or Formula (I), is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician.

The term “chemotherapy” refers to the treatment of cancer or a disease or disorder caused by a virus, bacterium, other microorganism, or an inappropriate immune response using specific chemical agents, drugs, or radioactive agents that are selectively toxic and destructive to malignant cells and tissues, viruses, bacteria, or other microorganisms. Chemotherapeutic agents or drugs such as an anti-folate (e.g., methotrexate) or any other agent or drug useful in treating cancer, an inflammatory disease, or an autoimmune disease are preferred. Suitable chemotherapeutic agents and drugs include, but are not limited to, actinomycin D, adriamycin, altretamine, azathioprine, bleomycin, busulphan, capecitabine, carboplatin, carmustine, chlorambucil, cisplatin, cladribine, crisantaspase, cyclophosphamide, cytarabine, dacarbazine, daunorubicin, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, methotrexate, mitomycin, mitozantrone, oxaliplatin, paclitaxel, pentostatin, procarbazine, raltitrexed, steroids, streptozocin, taxol, taxotere, temozolomide, thioguanine, thiotepa, tomudex, topotecan, treosulfan, uft (uracil-tegufur), vinblastine, vincristine, vindesine, and vinorelbine. Methotrexate is especially preferred.

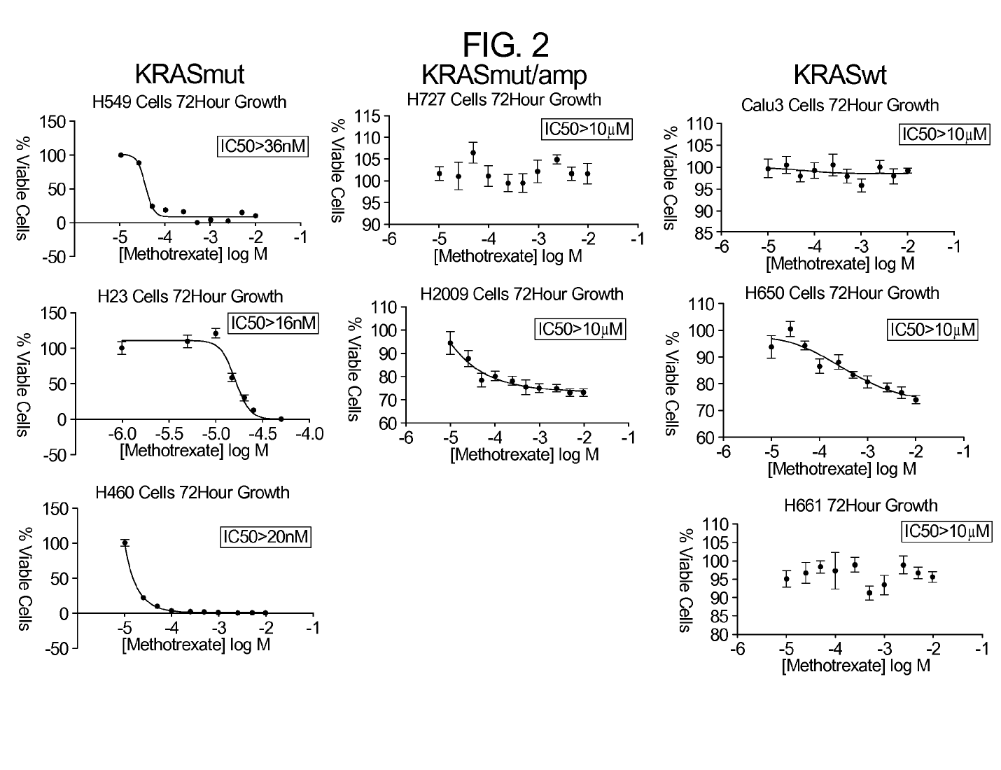

The term “methotrexate” is synonymous with “MTX” and refers to a molecule having the structure shown in FIG. 2, upper panel. Methotrexate includes, in part, a 2,4-diamino substituted pterine ring moiety linked at the 6 position to the amino group of a p-aminobenzoyl moiety, the p-aminobenzoyl moiety having a methylated amino group and being amide bonded to a glutamic acid moiety. As used herein, “MTXPG₁” is synonymous with methotrexate.

The term “methotrexate polyglutamate” is synonymous with “MTXPG” and refers to a derivative of methotrexate having two or more glutamates which are amide bonded to the p-aminobenzoyl moiety of methotrexate as shown in the generalized structure of FIG. 2, lower panel. The number of glutamates in a methotrexate polyglutamate varies from two to seven or more; the number of glutamate moieties can be denoted by “n” using the nomenclature MTXPGₙ such that, for example, MTXPG₂ is MTXPG having two glutamates, MTXPG₃ is MTXPG having three glutamates, MTXPG₄ is MTXPG having four glutamates, MTXPG₅ (SEQ ID NO:12) is MTXPG having five glutamates, MTXPG₆ (SEQ ID NO:15) is MTXPG having six glutamates, MTXPG₇ (SEQ ID NO:14) is MTXPG having seven glutamates, and MTXPG₂₋₇ (SEQ ID NO:11) is a mixture containing MTXPG₂, MTXPG₃, MTXPG₄, MTXPG₅ (SEQ ID NO:12), MTXPG₆ (SEQ ID NO:15), and MTXPG₇ (SEQ ID NO:14), with the ratio of the individual polyglutamated forms in the mixture not defined. As used herein, the term “long-chain MTXPG” refers to any MTX having at least three glutamates attached thereto (e.g., MTXPG₃).

The term “autoimmune disease” refers to a disease or disorder resulting from an immune response against a self tissue or tissue component and includes a self antibody response or cell-mediated response. The term autoimmune disease, as used herein, encompasses organ-specific autoimmune diseases, in which an autoimmune response is directed against a single tissue, such as Crohn's disease and ulcerative colitis, Type I diabetes mellitus, myasthenia gravis, vitiligo, Graves' disease, Hashimoto's disease, Addison's disease and autoimmune gastritis; and autoimmune hepatitis. The term autoimmune disease also encompasses non-organ specific autoimmune diseases, in which an autoimmune response is directed against a component present in several or many organs throughout the body. Such autoimmune diseases include, for example, rheumatoid disease, systemic lupus erythematosus, progressive systemic sclerosis and variants, polymyositis and dermatomyositis. Additional autoimmune diseases include, but are not limited to, pernicious anemia, autoimmune gastritis, primary biliary cirrhosis, autoimmune thrombocytopenia, Sjögren's syndrome, multiple sclerosis and psoriasis. One skilled in the art appreciates that the autoimmune diseases set forth above have been treated with chemotherapy such as methotrexate therapy and further recognizes that the methods of the invention can be used to optimize clinical responsiveness to the chemotherapy in a human or other mammal having any of the above or another autoimmune disease.

In some embodiments, a Ras mutation may comprise one or more mutations of V-Ki-ras2 Kirsten rat sarcoma viral oncogene homolog (k-Ras) (SEQ ID NO: 1), n-Ras (SEQ ID NO: 2) or h-Ras (SEQ ID NO: 3). Alternatively, a Ras mutation may be a variant including, for example, a biologically active variant, of the amino acid sequence as set forth in SEQ ID NO: 1, 2, or 3.

In some embodiments a Ras amplification may comprise one or more amplifications of k-Ras (SEQ ID NO: 4), n-Ras (SEQ ID NO: 5) or h-Ras (SEQ ID NO: 6). Alternatively, a Ras amplification may be a variant including, for example, a biologically active variant, of the nucleotide sequence as set forth in SEQ ID NO: 4, 5 or 6.

Guidance in determining which nucleotides or amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of Ras can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Thus, the polypeptides of the present invention may include one or more amino acid substitutions, deletions or additions, either from natural mutations or human manipulation.

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith, et al., J. Mol. Biol. 224:899-904 (1992) and de Vos, et al. Science 255:306-312 (1992)).

Variants of the Ras gene may include a polynucleotide possessing a nucleotide sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to Ras. Variants of Ras may include a polypeptide possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to Ras. Preferably, this variant may possess at least one biological property in common with the native protein.

Sequence identity or percent identity is intended to mean the percentage of the same residues shared between two sequences, when the two sequences are aligned using the Clustal method [Higgins et al, Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoff, et al., in Atlas of Protein Sequence and Structure, Dayhoff, Ed., NDRF, Washington, Vol. 5, suppl. 3, p. 345, (1978)].

In one embodiment, the disease or disorder may be cancer. In one embodiment the cancer may be selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma; parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment the cancer may be non-small cell lung cancer, pancreatic cancer, breast cancer, ovarian cancer, colorectal cancer, or head and neck cancer. In yet another embodiment the cancer may be a carcinoma, a tumor, a neoplasm, a lymphoma, a melanoma, a glioma, a sarcoma, or a blastoma.

In one embodiment the carcinoma may be selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiatied carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment the tumor may be selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In yet another embodiment the neoplasm may be selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment the lymphoma may be selected from the group consisting of: nervous system lymphoma, AIDS-related lymphoma, cutaneous T-cell lymphoma, non-Hodgkin's lymphoma, lymphoma, and Waldenstrom's macroglobulinemia. In another embodiment the melanoma may be selected from the group consisting of: acral lentiginous melanoma, superficial spreading melanoma, uveal melanoma, lentigo maligna melanomas, melanoma, intraocular melanoma, adenocarcinoma nodular melanoma, and hemangioma. In yet another embodiment the sarcoma may be selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment the glioma may be selected from the group consisting of: glioma, brain stem glioma, and hypothalamic and visual pathway glioma. In another embodiment the blastoma may be selected from the group consisting of: pulmonary blastoma, pleuropulmonary blastoma, retinoblastoma, neuroblastoma, medulloblastoma, glioblastoma, and hemangiblastomas.

Biological samples or test cells that may be used in the methods of the present disclosure may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, serum, lymph fluid, urine and faeces, or tissue which has been removed from organs, such as breast, lung, intestine, skin, cervix, prostate, and stomach. Biological samples may also include sections of tissues such as frozen sections taken for histological purposes.

### Methotrexate

Methotrexate is well known in the art as an inhibitor of dihydrofolate reductase (DHFR), which acts to decrease production of tetrahydrofolate (THF) from dihydrofolate (DHF). As a consequence, methotrexate indirectly inhibits purine and thymidine synthesis and amino acid interconversion. Methotrexate also exhibits anti-proliferative activity through inhibition of thymidylate synthesis, which is required to synthesize DNA (Calvert, Semin. Oncol. 26:3-10 (1999)). Methotrexate, its synthesis, and its properties are described in further detail in U.S. Pat. Nos. 2,512,572; 3,892,801; 3,989,703; 4,057,548; 4,067,867; 4,079,056; 4,080,325; 4,136,101; 4,224,446; 4,306,064; 4,374,987; 4,421,913; and 4,767,859. Methods of using methotrexate to treat cancer are described, for example, in U.S. Pat. Nos. 4,106,488, 4,558,690, and 4,662,359.

Methotrexate, which is useful in the treatment of a variety of autoimmune diseases and cancers, can be administered by oral or parenteral routes. The drug is readily distributed to body tissues, where it is transported into cells by a specific carrier system that includes components such as the reduced folate carrier, RFC-1, and the folate receptor. Due to its high polarity at physiological pH, methotrexate does not readily pass through the cell membrane, and the majority of the drug enters cells via specific carriers. Once inside the cell, methotrexate is converted to methotrexate polyglutamates by specific enzymes such as folylpolygamma-glutamate synthetase, which add one or more glutamic acid moieties, linked by iso-peptidic bonds to the γ-carboxyl of methotrexate as described, for example, in Kamen, Semin. Oncol. S18:30-39 (1997).

Detection of Ras Mutation and/or Amplification

A number of methodologies may be employed to detect the presence or absence including quantitating the expression (i.e., expression level or amount) of mutated Ras (e.g., one or more mutations in k-Ras, (SEQ ID NO: 1), n-Ras (SEQ ID NO: 2), or h-Ras (SEQ ID NO: 3) and/or the presence or absence of an amplification (e.g., 3, 4, 5, 6, 7, 8, 9, 10, 11, 12, 13, 14, 15, 16, 17, 18, 19, 20 or more copies per cell) of a Ras gene including, k-Ras, (SEQ ID NO: 4), n-Ras (SEQ ID NO: 5), or h-Ras (SEQ ID NO: 6) in a cell and/or a biological sample. Such detection of mutated Ras and/or amplification of Ras may be detected at the protein level and/or nucleic acid level. Those skilled in the art will appreciate that the methods indicated below represent some of the preferred ways in which the presence or absence, including the expression, of mutated Ras and/or the presence or absence of a Ras amplification may be detected and/or quantitated and in no manner limit the scope of methodologies that may be employed. Those skilled in the art will also be able to determine operative and optimal assay conditions for each determination by employing routine experimentation. Such methods may include but are not limited to in situ hybridization (ISH), Western blots, ELISA, immunoprecipitation, immunofluorescence, flow cytometry, northern blots, PCR, and immunocytochemistry (IHC). Ras may comprise the amino acid sequence set forth in SEQ ID NOS 1, 2 or 3. Alternatively, Ras may be a variant of the amino acid sequence as set forth in SEQ ID NOS 1, 2 or 3. A Ras amplification may comprise one or more amplifications of k-Ras (SEQ ID NO: 4), n-Ras (SEQ ID NO: 5) or h-Ras (SEQ ID NO: 6). Alternatively, a Ras amplification may be a variant including, for example, a biologically active variant, of the nucleotide sequence as set forth in SEQ ID NO: 4, 5 or 6.

In another embodiment, the methods may further involve obtaining a control sample and detecting mutated Ras and/or amplification of Ras in this control sample, such that the presence or absence mutated Ras and/or amplification of Ras in the control sample is determined. A negative control sample is useful if there is an absence of mutated Ras and/or amplification of Ras, whereas a positive control sample is useful if there is a presence of mutated Ras and/or amplification of Ras. For the negative control, the sample may be from the same individual as the test sample (i.e. different location such as tumor versus non-tumor) or may be from a different individual known to have an absence of mutated Ras and/or amplification of Ras.

A biological sample may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, lymph fluid, urine and faeces, or tissue which has been removed from organs, such as breast, lung, intestine, skin, cervix, prostate, and stomach.

### Detection/Quantitation of Ras Mutation

In an embodiment, the mutated Ras may be detected at the nucleic acid or protein level. Nucleic acid-based techniques for assessing expression are well known in the art and include, for example, determining the level of Ras mRNA in a biological sample. Many expression detection methods use isolated RNA. Any RNA isolation technique that does not select against the isolation of mRNA can be utilized for the purification of RNA from cervical cells (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

Isolated mRNA from a biological sample can be used in hybridization or amplification assays that include, but are not limited to, Southern or Northern analyses, polymeRase chain reaction analyses and probe arrays. One method for the detection of Ras mRNA levels involves contacting the isolated mRNA with a nucleic acid molecule (probe) that can hybridize to the mRNA encoded by the Ras gene. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding Ras. Hybridization of an mRNA with the probe indicates that Ras is being expressed.

In one embodiment, the mRNA from a biological sample is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array.

An alternative method for determining the level of Ras mRNA in a biological sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, biomarker expression may be assessed by quantitative fluorogenic RT-PCR (i.e., the TaqMan® System). Such methods typically may utilize pairs of oligonucleotide primers that are specific for Ras. Methods for designing oligonucleotide primers specific for a known sequence are well known in the art.

Expression levels of Ras RNA may be monitored using a membrane blot (such as used in hybridization analysis such as Northern, Southern, dot, and the like), or microwells, sample tubes, gels, beads or fibers (or any solid support comprising bound nucleic acids) (see, e.g., U.S. Pat. Nos. 5,770,722, 5,874,219, 5,744,305, 5,677,195 and 5,445,934). The detection of Ras expression may also comprise using nucleic acid probes in solution.

In one embodiment of the disclosure, microarrays are used to detect Ras expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA may be hybridized to complementary probes on the array and then detected by laser scanning. Hybridization intensities for each probe on the array are determined and converted to a quantitative value representing relative gene expression levels (see, e.g., U.S. Pat. Nos. 6,040,138, 5,800,992, 6,020,135, 6,033,860, and 6,344,316). High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device (see, e.g., U.S. Pat. Nos. 5,856,174 and 5,922,591).

In one approach, total mRNA isolated from the biological sample may be converted to labeled cRNA and then hybridized to an oligonucleotide array. Each sample may be hybridized to a separate array. Relative transcript levels may be calculated by reference to appropriate controls present on the array and in the sample.

In a particular embodiment, the level of Ras mRNA can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (see, e.g., U.S. Pat. No. 4,843,155).

The isolated mRNA can be used in hybridization or amplification assays that include, but are not limited to, Southern or Northern analyses, polymeRase chain reaction analyses and probe arrays. One preferred diagnostic method for the detection of Ras mRNA levels involves contacting the isolated mRNA with a nucleic acid molecule (probe) that can hybridize to the Ras mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding Ras. Other suitable probes for use in the diagnostic assays of the disclosure are described herein. Hybridization of an mRNA with the probe indicates that Ras is being expressed.

In one format, the mRNA may be immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA may be contacted with the probe(s), for example, in an Affymetrix gene chip array. A skilled artisan can readily adapt known mRNA detection methods for use in detecting the level of mRNA encoded by Ras.

An alternative method for determining the level of Ras mRNA in a biological sample involves the process of nucleic acid amplification, e.g., by RT-PCR (see, e.g., U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample may be prepared/processed using known histological methods. The sample may be then immobilized on a support, typically a glass slide, and then contacted with a probe that can hybridize to Ras mRNA.

In another embodiment of the present disclosure, a Ras protein may be detected. A preferred agent for detecting Ras protein of the disclosure is an antibody capable of binding to such a protein or a fragment thereof, preferably an antibody with a detectable label. Antibodies can be polyclonal, or more preferably, monoclonal. An intact antibody, or a fragment or derivative thereof can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that may be directly labeled. Examples of indirect labeling include detection of a primary antibody using a fluorescently labeled secondary antibody and end-labeling of a DNA probe with biotin such that it can be detected with fluorescently labeled streptavidin.

Antibody fragments may comprise a portion of an intact antibody, preferably the antigen-binding or variable region of the intact antibody. Examples of antibody fragments include Fab, Fab', F(ab′)2, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and may be still capable of cross-linking antigen.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesteRase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include lucifeRase, luciferin, and aequorin; and examples of suitable radioactive material include ¹²⁵I, ¹³¹I, ³⁵S, or ³H.

In regard to detection of antibody staining in the immunocytochemistry methods of the disclosure, there also exist in the art, video-microscopy and software methods for the quantitative determination of an amount of multiple molecular species (e.g., biomarker proteins) in a biological sample wherein each molecular species present may be indicated by a representative dye marker having a specific color. Such methods are also known in the art as a colorimetric analysis methods. In these methods, video-microscopy may be used to provide an image of the biological sample after it has been stained to visually indicate the presence of a particular biomarker of interest. Some of these methods, such as those disclosed in U.S. patent application Ser. Nos. 09/957,446 and 10/057,729, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that may be deconstructed into its component color parts.

The antibodies used to practice the disclosure are selected to have high specificity for Ras including, for example, mutated Ras. Methods for making antibodies and for selecting appropriate antibodies are known in the art (see, e.g., Celis, ed. (in press) Cell Biology & Laboratory Handbook, 3rd edition (Academic Press, New York)). In some embodiments, commercial antibodies directed to specific Ras proteins may be used to practice the disclosure. The antibodies of the disclosure may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry may be needed to maximize the signal to noise ratio for a particular antibody. Antibody concentrations that maximize specific binding to Ras and minimize non-specific binding (or background) can be determined. In particular embodiments, appropriate antibody titers for use in cytology preparations are determined by initially testing various antibody dilutions on formalin-fixed paraffin-embedded normal and high-grade cervical disease tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody may be then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the disclosure will vary depending on such factors as time for binding, level of specificity of the antibody for Ras protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells express a biomarker of the present disclosure.

One skilled in the art will know many other suitable carriers for binding antibody or antigen, and will be able to adapt such support for use with the present disclosure. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies.

The present disclosure also includes methods for fixing cells and tissue samples for analysis. Generally, neutral buffered formalin may be used. Any concentration of neutral buffered formalin that can fix tissue or cell samples without disrupting the epitope can be used. In one embodiment a solution of about 10 percent may be used. Preferably, the method includes suitable amounts of phosphatase inhibitors to inhibit the action of phosphatases and preserve phosphorylation. Any suitable concentration of phosphatase inhibitor can be used so long as the biopsy sample is stable and phosphatases are inhibited, for example 1 mM NaF and/or Na3VO4 can be used. In one method a tissue sample or tumor biopsy may be removed from a patient and immediately immersed in a fixative solution which can and preferably does contain one or more phosphatase inhibitors, such as NaF and/or Na3VO4. Preferably, when sodium orthovanadate is used it is used in an activated or depolymerized form to optimize its activity.

Depolymerization can be accomplished by raising the pH of its solution to about 10 and boiling for about 10 minutes. The phosphatase inhibitors can be dissolved in the fixative just prior to use in order to preserve their activity.

Fixed samples can then be stored for several days or processed immediately. To process the samples into paraffin after fixing, the fixative can be thoroughly rinsed away from the cells by flushing the tissue with water. The sample can be processed to paraffin according to normal histology protocols which can include the use of reagent grade ethanol. Samples can be stored in 70% ethanol until processed into paraffin blocks. Once samples are processed into paraffin blocks they can be analyzed histochemically for virtually any antigen that is stable to the fixing process.

In preferred embodiments, Ras staining may be detected, measured and quantitated automatically using automated image analysis equipment. Such equipment can include a light or fluorescence microscope, and image-transmitting camera and a view screen, most preferably also comprising a computer that can be used to direct the operation of the device and store and manipulate the information collected, most preferably in the form of optical density of certain regions of a stained tissue preparation. Image analysis devices useful in the practice of this disclosure include but are not limited to the CAS 200 (Becton Dickenson, Mountain View, Calif.), Chromavision or Tripath systems. Using such equipment the quantity of the target epitope in unknown cell samples can be determined using any of a variety of methods that are known in the art. The cell pellets can be analyzed by eye such that the optical density reading of the control cells can be correlated to a manual score such as 0, 1+, 2+ or 3+, as in Table 1 below which shows the correlation between quantitative image analysis data measured in optical density (OD) and manual score.

Automated (computer-aided) image analysis systems known in the art can augment visual examination of biological samples. In a representative system, the cell or tissue sample may be exposed to detectably labeled reagents specific for Ras (e.g., mutated Ras), and the magnified image of the cell may be then processed by a computer that receives the image from a charge-coupled device (CCD) or camera such as a television camera. Such a system can be used, for example, to detect and measure expression and activation levels of Her1, pHER1 HER2, HER3, and pERK in a sample. Additional biomarkers are also contemplated by this disclosure. This methodology provides more accurate diagnosis of cancer and a better characterization of gene expression in histologically identified cancer cells, most particularly with regard to expression of tumor marker genes or genes known to be expressed in particular cancer types and subtypes (i.e., different degrees of malignancy). This information permits a more informed and effective regimen of therapy to be administered, because drugs with clinical efficacy for certain tumor types or subtypes can be administered to patients whose cells are so identified.

For example, expression and activation of Ras proteins expressed from tumor-related genes can be detected and quantitated using methods of the present disclosure. Further, expression and activation of proteins that are cellular components of a tumor-related signaling pathway can be detected and quantitated using methods of the present disclosure. Further, proteins associated with cancer can be quantified by image analysis using a suitable primary antibody against biomarkers, such as, but not limited to, Her-1, Her-2, p-Her-1, Her-3, or p-ERK, and a secondary antibody (such as rabbit anti-mouse IgG when using mouse primary antibodies) and/or a tertiary avidin (or Strepavidin) biotin complex (“ABC”).

In practicing the method of the present disclosure, staining procedures can be carried out by a technician in the laboratory. Alternatively, the staining procedures can be carried out using automated systems. In either case, staining procedures for use according to the methods of this disclosure are performed according to standard techniques and protocols well-established in the art.

The amount of Ras can then be quantitated by the average optical density of the stained antigens. Also, the proportion or percentage of total tissue area stained may be readily calculated, as the area stained above an antibody threshold level in the second image. Following visualization of nuclei containing Ras, the percentage or amount of such cells in tissue derived from patients after treatment may be compared to the percentage or amount of such cells in untreated tissue or said tissue prior to treatment.

### Detection/Quantitation of Ras Amplification

The present invention encompasses methods of gene amplification known to those of skill in the art, see, for example, Boxer, J. Clin. Pathol. 53: 19-21 (2000). Such techniques include in situ hybridization (Stoler, Clin. Lab. Med. 12:215-36 (1990), using radioisotope or fluorophore-labeled probes; polymerase chain reaction (PCR); quantitative Southern blotting, dot blotting and other techniques for quantitating individual genes. Preferably, probes or primers selected for gene amplification evaluation are highly specific, to avoid detecting closely related homologous genes. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

In one embodiment, the biological sample contains nucleic acids from the test subject. The nucleic acids may be mRNA or genomic DNA molecules from the test subject.

1. Amplification Based Assays

In one embodiment of the present invention, amplification-based assays can be used to measure copy number of the Ras gene. In such amplification-based assays, the corresponding Ras nucleic acid sequence acts as a template in an amplification reaction (for example, Polymerase Chain Reaction or PCR). In a quantitative amplification, the amount of amplification product will be proportional to the amount of template in the original sample. Comparison to appropriate controls provides a measure of the copy-number of the Ras gene, corresponding to the specific probe used. The presence of a higher level of amplification product, as compared to a control, is indicative of amplified Ras.

a. Quantitative PCR

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided, for example, in Innis et al. (1990) PCR Protocols, A Guide to Methods and Applications, Academic Press, Inc. N.Y. The known nucleic acid sequence for the Met (Accession No.: NM 000245) is sufficient to enable one of skill to routinely select primers to amplify any portion of the Ras gene.

b. Real Time PCR

Real time PCR is another amplification technique that can be used to determine gene copy levels or levels of Ras mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copiesof the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

A TaqMan-based assay also can be used to quantify MET polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification.

c. Other Amplification Methods

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

2. Hybridization Based Assays

Hybridization assays can be used to detect Ras copy number. Hybridization-based assays include, but are not limited to, traditional “direct probe” methods such as Southern blots or in situ hybridization (e.g., FISH), and “comparative probe” methods such as comparative genomic hybridization (CGH). The methods can be used in a wide variety of formats including, but not limited to substrate—(e.g. membrane or glass) bound methods or array-based approaches as described below.

a. Southern Blot

One method for evaluating the copy number of Ras encoding nucleic acid in a sample involves a Southern transfer. Methods for doing Southern Blots are known to those of skill in the art (see Current Protocols in Molecular Biology, Chapter 19, Ausubel, et al., Eds., Greene Publishing and Wiley-Interscience, New York, 1995, or Sambrook et al., Molecular Cloning: A Laboratory Manual, 2d Ed. vol. 1-3, Cold Spring Harbor Press, NY, 1989). In such an assay, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. An intensity level that is higher than the control is indicative of amplified Ras.

b. Fluorescence In Situ Hybridization (FISH)

In another embodiment, FISH is used to determine the copy number of the Ras gene in a sample. Fluorescence in situ hybridization (FISH) is known to those of skill in the art (see Angerer, 1987 Meth. Enzymol., 152: 649). Generally, in situ hybridization comprises the following major steps: (1) fixation of tissue or biological structure to be analyzed; (2) pre-hybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

In some applications it is necessary to block the hybridization capacity of repetitive sequences. Thus, in some embodiments, tRNA, human genomic DNA, or Cot-1 DNA is used to block non-specific hybridization. Thus, in one embodiment of the present invention, the presence or absence of Ras amplification is determined by FISH.

c. Comparative Genomic Hybridization (CGH)

In comparative genomic hybridization methods, a “test” collection of nucleic acids (e.g. from a possible tumor) is labeled with a first label, while a second collection (e.g. from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection, is detected and the ratio provides a measure of the gene copy number, corresponding to the specific probe used. A cytogenetic representation of DNA copy-number variation can be generated by CGH, which provides fluorescence ratios along the length of chromosomes from differentially labeled test and reference genomic DNAs. In another embodiment of the present invention, comparative genomic hybridization may be used to detect the presence or absence of Ras amplification.

d. Microarray Based Comparative Genomic Hybridization

In an alternative embodiment of the present invention, DNA copy numbers are analyzed via microarray-based platforms. Microarray technology offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068; Pollack et al., Nat. Genet., 23(1):41-6, (1999), Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211 and others.

The DNA used to prepare the arrays of the invention is not critical. For example, the arrays can include genomic DNA, e.g. overlapping clones that provide a high resolution scan of a portion of the genome containing the desired gene, or of the gene itself. Genomic nucleic acids can be obtained from, e.g., HACs, MACs, YACs, BACs, PACs, P1 s, cosmids, plasmids, inter-Alu PCR products of genomic clones, restriction digests of genomic clones, cDNA clones, amplification (e.g., PCR) products, and the like. Arrays can also be produced using oligonucleotide synthesis technology. Thus, for example, U.S. Pat. No. 5,143,854 and PCT Patent Publication Nos. WO 90/15070 and WO 92/10092 teach the use of light-directed combinatorial synthesis of high density oligonucleotide arrays.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), Pinkel et al. (1998) Nature Genetics 20: 207-211, or of Kallioniemi (1992) Proc. Natl. Acad Sci USA 89:5321-5325 (1992), etc.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

In another embodiment of the present invention, kits useful for the detection of Met amplification are disclosed. Such kits may include any or all of the following: assay reagents, buffers, specific nucleic acids or antibodies (e.g. full-size monoclonal or polyclonal antibodies, single chain antibodies (e.g., scFv), or other gene product binding molecules), and other hybridization probes and/or primers, and/or substrates for polypeptide gene products.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Methods for Predicting Sensitivity to a Drug

The present disclosure provides methods for predicting sensitivity of a test cell to a DHFR inhibitor, by obtaining a test cell; assaying the test cell for one or more Ras mutations (e.g., one or more mutations in k-Ras (SEQ ID NO: 1), n-Ras (SEQ ID NO: 2) or h-Ras (SEQ ID NO: 3); determining if one or more Ras mutations are present or absent (e.g., Ras wild type) in the test cell; and employing the determination of the presence or absence of a Ras mutation in the test cell to predict sensitivity of the test cell to the drug. In some embodiments, the test cell is predicted to be sensitive to the DHFR inhibitor where one or more Ras mutations are determined to be present in the test cell. In some embodiments, the test cell is predicted to be sensitive to the DHFR inhibitor where Ras mutations are determined to be absent in the test cell. In some embodiments, the test cell is predicted to be insensitive to the DHFR inhibitor where one or more Ras mutations are determined to be present in the test cell. In some embodiments, the test cell is predicted to be insensitive to the DHFR inhibitor where one or more Ras mutations are determined to be absent in the test cell.

The present disclosure provides methods for predicting sensitivity of a test cell (e.g., a cell obtained from a cancer patient) to a drug (e.g., an antifolate such as a dihydrofolate reductase (DHFR); or an EGFR inhibitor) by obtaining a test cell; assaying the test cell for one or more Ras mutations (e.g., one or more mutations in k-Ras (SEQ ID NO: 1), n-Ras (SEQ ID NO: 2) or h-Ras (SEQ ID NO: 3); assaying the test cell for amplification of a Ras gene; determining if one or more Ras mutations are present or absent in the test cell and determining if an amplification of the Ras gene is present or absent in the test cell; and employing the determination of the presence or absence of a Ras mutation in the test cell and the presence or absence of an amplification of Ras in the test cell to predict sensitivity of the test cell to the drug. In some embodiments, the test cell is predicted to be sensitive to the drug where one or more Ras mutations are determined to be present in the test cell and an amplification of Ras is determined to be present in the test cell. In some embodiments, the test cell is predicted to be sensitive to the drug where one or more Ras mutations are determined to be present in the test cell and amplification of Ras is determined to be absent in the test cell. In some embodiments, the test cell is predicted to be sensitive to the drug where Ras mutations are determined to be absent (e.g., Ras wild type) in the test cell and an amplification of Ras is determined to be present in the test cell. In some embodiments, the test cell is predicted to be sensitive to the drug where Ras mutations are determined to be absent (e.g., Ras wild type) in the test cell and amplification of Ras is determined to be absent in the test cell. In some embodiments, the test cell is predicted to be insensitive to the drug where one or more Ras mutations are determined to be present in the test cell and an amplification of Ras is determined to be present in the test cell. In some embodiments, the test cell is predicted to be insensitive to the drug where one or more Ras mutations are determined to be present in the test cell and amplification of Ras is determined to be absent in the test cell. In some embodiments, the test cell is predicted to be insensitive to the drug where Ras mutations are determined to be absent (e.g., Ras wild type) in the test cell and an amplification of Ras is determined to be present in the test cell. In some embodiments, the test cell is predicted to be insensitive to the drug where Ras mutations are determined to be absent (e.g., Ras wild type) in the test cell and amplification of Ras is determined to be absent in the test cell.

In some embodiments, the test cell is predicted to be sensitive to the drug where the number of Ras mutations in the test cell is elevated as compared to the number of Ras mutations in a control cell or is above a threshold. In some embodiments, the test cell is predicted to be sensitive to the drug where the number of Ras mutations in the test cell is reduced as compared to the number of Ras mutations in a control cell or is above a threshold. In some embodiments, the test cell is predicted to be sensitive to the drug where the number of amplifications of Ras in the test cell is elevated as compared to the number of amplifications of Ras in a control cell or is above a threshold. In some embodiments, the test cell is predicted to be sensitive to the drug where the number of amplifications of Ras in the test cell is reduced as compared to the number of amplifications of Ras in a control cell or is above a threshold. In some embodiments, the test cell is predicted to be sensitive to the drug where the number of Ras mutations is elevated and number of amplifications of Ras in the test cell is elevated as compared to the number of Ras mutations and number of amplifications of Ras in a control cell or is above a threshold. In some embodiments, the test cell is predicted to be sensitive to the drug where the number of Ras mutations is reduced and number of amplifications of Ras in the test cell is elevated as compared to the number of Ras mutations and number of amplifications of Ras in a control cell or is above a threshold. In some embodiments, the test cell is predicted to be sensitive to the drug where the number of Ras mutations is elevated and number of amplifications of Ras in the test cell is reduced as compared to the number of Ras mutations and number of amplifications of Ras in a control cell or is above a threshold. In some embodiments, the test cell is predicted to be insensitive to the drug where the number of Ras mutations in the test cell is elevated as compared to the number of Ras mutations in a control cell or is above a threshold. In some embodiments, the test cell is predicted to be insensitive to the drug where the number of Ras mutations in the test cell is reduced as compared to the number of Ras mutations in a control cell or is above a threshold. In some embodiments, the test cell is predicted to be insensitive to the drug where the number of amplifications of Ras in the test cell is elevated as compared to the number of amplifications of Ras in a control cell or is above a threshold. In some embodiments, the test cell is predicted to be insensitive to the drug where the number of amplifications of Ras in the test cell is reduced as compared to the number of amplifications of Ras in a control cell or is above a threshold. In some embodiments, the test cell is predicted to be insensitive to the drug where the number of Ras mutations is elevated and number of amplifications of Ras in the test cell is elevated as compared to the number of Ras mutations and number of amplifications of Ras in a control cell or is above a threshold. In some embodiments, the test cell is predicted to be insensitive to the drug where the number of Ras mutations is reduced and number of amplifications of Ras in the test cell is elevated as compared to the number of Ras mutations and number of amplifications of Ras in a control cell or is above a threshold. In some embodiments, the test cell is predicted to be insensitive to the drug where the number of Ras mutations is elevated and number of amplifications of Ras in the test cell is reduced as compared to the number of Ras mutations and number of amplifications of Ras in a control cell or is above a threshold.

In some embodiments, the threshold may be set at a number of Ras mutations and/or level of expression of mutated Ras and/or number of Ras amplifications above which a control cell is known to be sensitive to treatment with the drug and below which the control cell is known to not be sensitive to treatment with the drug.

In some embodiments, the threshold is set at a number of Ras mutations and/or level of expression of mutated Ras and/or number of Ras amplifications above which 50%, 60%, 70%, 80%, 90%, or 95% of control cells respond to treatment with the drug.

In some embodiments, the threshold is set at a number of Ras mutations and/or level of expression of mutated Ras and/or number of Ras amplifications below which 50%, 60%, 70%, 80%, 90%, or 95% of control cells do not respond to treatment with the drug.

### Methods for Predicting Responsiveness of a Subject to a Drug

The present disclosure also provides methods for predicting responsiveness of a subject with a disease or disorder to treatment with a DHFR inhibitor by obtaining a biological sample from the subject; assaying target cells obtained from the biological sample for one or more Ras mutations (e.g., one or more mutations in k-Ras (SEQ ID NO: 1), n-Ras (SEQ ID NO: 2) or h-Ras (SEQ ID NO: 3); determining if one or more Ras mutations are present or absent (e.g., Ras wild type) in the target cells; and employing the determination of the presence or absence of a Ras mutation in the target cells obtained from the biological sample to predict responsiveness of the subject to the DHFR inhibitor. In some embodiments, the subject is predicted to be responsive to the DHFR inhibitor where one or more Ras mutations are present in the target cells. In some embodiments, the subject is predicted to be responsive to the DHFR inhibitor where one or more Ras mutations are absent in the target cells. In some embodiments, the subject is predicted to be non-responsive to the DHFR inhibitor where one or more Ras mutations are present in the target cells. In some embodiments, the subject is predicted to be non-responsive to the DHFR inhibitor where one or more Ras mutations are absent in the target.

The present disclosure also provides methods for predicting responsiveness of a subject with a disease or disorder to treatment with a drug (e.g., an antifolate such as a dihydrofolate reductase (DHFR); or an EGFR inhibitor) by obtaining a biological sample (e.g., a biological sample obtained from a cancer patient such as a formalin fixed paraffin embedded tissue) from the subject; assaying target cells obtained from the biological sample for one or more Ras mutations (e.g., one or more mutations in k-Ras (SEQ ID NO: 1), n-Ras (SEQ ID NO: 2) or h-Ras (SEQ ID NO: 3); assaying target cells obtained from the biological sample for a Ras amplification; determining if one or more Ras mutations are present or absent in the target cells and determining if an amplification of the Ras gene is present or absent in the target cells; and employing the determination of the presence or absence of a Ras mutation and the presence or absence of an amplification of Ras in the target cells obtained from the biological sample to predict responsiveness of the subject to the drug. In some embodiments, the subject is predicted to be responsive to the drug where one or more Ras mutations are present in the target cells and an amplification of Ras is present in the target cells. In some embodiments, the subject is predicted to be responsive to the drug where one or more Ras mutations are absent (e.g., Ras wild type) in the target cells and an amplification of Ras is present in the target cells. In some embodiments, the subject is predicted to be responsive to the drug where one or more Ras mutations are present in the target cells and an amplification of Ras is absent in the target cells. In some embodiments, the subject is predicted to be responsive to the drug where one or more Ras mutations are absent (e.g., Ras wild type) in the target cells and an amplification of Ras is absent in the target cells. In some embodiments, the subject is predicted to be non-responsive to the drug where one or more Ras mutations are present in the target cells and an amplification of Ras is present in the target cells. In some embodiments, the subject is predicted to be non-responsive to the drug where one or more Ras mutations are absent (e.g., Ras wild type) in the target cells and an amplification of Ras is present in the target cells. In some embodiments, the subject is predicted to be non-responsive to the drug where one or more Ras mutations are present in the target cells and an amplification of Ras is absent in the target cells. In some embodiments, the subject is predicted to be non-responsive to the drug where one or more Ras mutations are absent (e.g., Ras wild type) in the target cells and an amplification of Ras is absent in the target cells.

The subject may be predicted to be responsive to the drug where the number of Ras mutations and/or number of Ras amplifications in the biological sample is elevated as compared to the control sample or is greater than a threshold. Alternatively, the subject may be predicted to not be responsive to the drug where the number of Ras mutations and/or number of Ras amplifications in the biological sample is reduced as compared to the control sample or is less than the threshold. The threshold may be set at a number of Ras mutations and/or number of Ras amplifications above which the control sample is known to respond to treatment with the drug and below which a control sample is known to not respond to treatment with the drug. In some embodiments, the threshold may be set at the number of Ras mutations and/or number of Ras amplifications above which 50%, 60%, 70%, 80%, 90%, or 95% of control samples respond to treatment with a drug and/or at a number of Ras mutations and/or number of Ras amplifications below which 50%, 60%, 70%, 80%, 90%, or 95% of control samples do not respond to treatment with a drug. Alternatively, a subject may be predicted to be responsive to a drug where the expression (e.g., amount or level) of mutant Ras and/or the number of Ras amplifications detected in the biological sample is above or below a set threshold. For example, a threshold may be set at the maximum amount of expression of mutated Ras and/or number of Ras amplifications in a biological sample obtained from a subject where the subject is responsive to treatment with a drug. Such a threshold may be an average or median obtained from two or more subjects.

In some embodiments, the subject may be predicted to be responsive to a drug where the number of Ras mutations and/or level of mutant Ras expression and/or number of Ras amplifications in a biological sample (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the number of Ras mutations and/or level of mutant Ras expression and/or the number of Ras amplifications detected in a control sample. In some embodiments, the subject may be predicted to be responsive to a drug where the number of Ras mutations and/or level of mutant Ras expression and/or number of Ras amplifications in a biological sample (e.g., tumor cells in the biological sample) is 2 times, 3 times, 4 times, 5 times, 6 times, 7 times, 8 times, 9 times, 10 times or more than the number of Ras mutations and/or level of mutant Ras expression and/or number of Ras amplifications in a biological sample detected in a control sample. In some embodiments, the biological sample and control sample are from the same specimen. In some embodiments, the biological sample and control sample are from the different specimens.

In some embodiments, the threshold may be set at a number of Ras mutations and/or level of expression of mutated Ras and/or number of Ras amplifications above which a control cell is known to be sensitive to treatment with the drug and below which the control cell is known to not be sensitive to treatment with the drug.

In some embodiments, the threshold is set at a number of Ras mutations and/or level of expression of mutated Ras and/or number of Ras amplifications above which 50%, 60%, 70%, 80%, 90%, or 95% of control cells respond to treatment with the drug.

In some embodiments, the threshold is set at a number of Ras mutations and/or level of expression of mutated Ras and/or number of Ras amplifications below which 50%, 60%, 70%, 80%, 90%, or 95% of control cells do not respond to treatment with the drug.

### Pharmaceutical Formulations

Pharmaceutical formulations comprising one or more drugs including, for example, chemotherapeutic agents are provided. Such agents may include an antifolate including, for example, a dihydrofolate reductase (DHFR) inhibitor such as Methotrexate or Pemetrexed. Such agents may additionally or alternatively include a tyrosine kinase inhibitor that targets HER1 (EGFR), HER2/neu, HER3, or any combination thereof such as cetuximab (Erbitux), panitumumab, zalutumumab, nimotuzumab or matuzumab.

The drug can be administered as an active ingredient in admixture with suitable pharmaceutical diluents, excipients, or carriers (collectively referred to herein as “carrier” materials) suitably selected with respect to the intended form of administration, that is, oral tablets, capsules, elixirs, syrups and the like, and consistent with conventional pharmaceutical practices.

For example, in one embodiment, the pharmaceutical composition comprises a drug solution with L-arginine. To prepare this composition, a 10 g quantity of L-arginine was added to a vessel containing approximately 70 mL of Water-For-Injections BP. The mixture was stirred with a magnetic stirrer until the arginine had dissolved. A 5 g quantity of PXD-101 was added, and the mixture stirred at 25° C. until the PXD-101 had dissolved. The solution was diluted to a final volume of 100 mL using Water-For-Injections BP. The resulting solution had a pH of 9.2-9.4 and an osmolality of approximately 430 mOSmol/kg. The solution was filtered through a suitable 0.2 sterilizing (e.g., PVDF) membrane. The filtered solution was placed in vials or ampoules, which were sealed by heat, or with a suitable stopper and cap. The solutions were stored at ambient temperature, or, more preferably, under refrigeration (e.g., 2-8° C.) in order to reduced degradation of the drug.

In one embodiment, the drug can be administered orally. Oral administration can be in the form of a tablet or capsule. The drug can be combined with an oral, non-toxic, pharmaceutically acceptable, inert carrier such as lactose, starch, sucrose, glucose, methyl cellulose, microcrystalline cellulose, sodium croscarmellose, magnesium stearate, dicalcium phosphate, calcium sulfate, mannitol, sorbitol and the like or a combination thereof. For oral administration in liquid form, the drug can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, microcrystalline cellulose, sodium croscarmellose, polyethylene glycol, waxes and the like. Lubricants suitable for use in these dosage forms include sodium oleate, sodium stearate, magnesium stearate, sodium benzoate, sodium acetate, sodium chloride, and the like. Disintegrators suitable for use in these dosage forms include starch methyl cellulose, agar, bentonite, xanthan gum and the like.

Suitable pharmaceutically acceptable salts of the drugs described herein, and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N1N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

Various further aspects and embodiments of the present invention will be apparent to those skilled in the art in view of the present disclosure. All documents and database entries mentioned in this specification are incorporated herein by reference in their entirety. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The drug can be administered in an oral form, for example, as tablets, capsules (each of which includes sustained release or timed release formulations), pills, powders, granules, elixirs, tinctures, suspensions, syrups, and emulsions, all well known to those of ordinary skill in the pharmaceutical arts. Likewise, the drug can be administered in intravenous (bolus or infusion), intraperitoneal, subcutaneous, or intramuscular form, well known to those of ordinary skill in the pharmaceutical arts.

The drug can be administered in the form of a depot injection or implant preparation that can be formulated in such a manner as to permit a sustained release of the active ingredient. The active ingredient can be compressed into pellets or small cylinders and implanted subcutaneously or intramuscularly as depot injections or implants. Implants can employ inert materials such as biodegradable polymers or synthetic silicones, for example, Silastic, silicone rubber or other polymers manufactured by the Dow-Corning Corporation.

The drug can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines.

The drug can also be delivered by the use of monoclonal antibodies as individual carriers to which the compound molecules are coupled.

The drug can also be prepared with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues.

Furthermore, the drug can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and cross linked or amphipathic block copolymers of hydrogels. The dosage regimen utilizing the drug can be selected in accordance with a variety of factors including type, species, age, weight, sex and the type of cancer being treated; the severity (i.e., stage) of the cancer to be treated; the route of administration; the renal and hepatic function of the subject; and the particular compound or salt thereof employed. An ordinarily skilled physician or veterinarian can readily determine and prescribe the effective amount of the drug required to treat, for example, to prevent, inhibit (fully or partially) or arrest the progress of the disease.

Oral dosages of the drug, when used to treat the desired cancer can range between about 2 mg to about 6000 mg per day, such as from about 20 mg to about 6000 mg per day, such as from about 200 mg to about 6000 mg per day. For example, oral dosages can be about 2, about 20, about 200, about 400, about 800, about 1200, about 1600, about 2000, about 4000, about 5000 or about 6000 mg per day. It is understood that the total amount per day can be administered in a single dose or can be administered in multiple dosing such as twice, three or four times per day.

For example, a subject can receive between about 2 mg/day to about 2000 mg/day, for example, from about 20 to about 2000 mg/day, such as from about 200 to about 2000 mg/day, for example from about 400 mg/day to about 1200 mg/day. A suitably prepared medicament for once a day administration can thus contain between about 2 mg and about 2000 mg, such as from about 20 mg to about 2000 mg, such as from about 200 mg to about 1200 mg, such as from about 400 mg/day to about 1200 mg/day. The drug can be administered in a single dose or in divided doses of two, three, or four times daily. For administration twice a day, a suitably prepared medicament would therefore contain half of the needed daily dose.

Intravenously or subcutaneously, the subject would receive the drug in quantities sufficient to deliver between about 3-1500 mg/m2 per day, for example, about 3, 30, 60, 90, 180, 300, 600, 900, 1000, 1200, or 1500 mg/m2 per day. Such quantities can be administered in a number of suitable ways, e.g., large volumes of low concentrations of drug during one extended period of time or several times a day. The quantities can be administered for one or more consecutive days, intermittent days, or a combination thereof per week (7 day period). Alternatively, low volumes of high concentrations of drug during a short period of time, e.g., once a day for one or more days either consecutively, intermittently, or a combination thereof per week (7 day period). For example, a dose of 300 mg/m2 per day can be administered for 5 consecutive days for a total of 1500 mg/m2 per treatment. In another dosing regimen, the number of consecutive days can also be 5, with treatment lasting for 2 or 3 consecutive weeks for a total of 3000 mg/m2 and 4500 mg/m2 total treatment.

Typically, an intravenous formulation can be prepared which contains a concentration of drug of from about 1.0 mg/mL to about 10 mg/mL, e.g., 2.0 mg/mL, 3.0 mg/mL, 4.0 mg/mL, 5.0 mg/mL, 6.0 mg/mL, 7.0 mg/mL, 8.0 mg/mL, 9.0 mg/mL, or 10 mg/mL, and administered in amounts to achieve the doses described above. In one example, a sufficient volume of intravenous formulation can be administered to a subject in a day such that the total dose for the day is between about 300 and about 1200 mg/m2.

In a preferred embodiment, 1000 mg/m2 of PXD-101 is administered intravenously once daily by 30-minute infusion every 24 hours for at least five consecutive days.

In one embodiment, PXD-101 is administered in a total daily dose of up to 1500 mg/m2. In one embodiment, PXD-101 is administered intravenously in a total daily dose of 1000 mg/m2, or 1400 mg/m2 or 1500 mg/m2, for example, once daily, continuously (every day), or intermittently. In one embodiment, PXD-101 is administered every day on days 1 to 5 every three weeks.

Glucuronic acid, L-lactic acid, acetic acid, citric acid, or any pharmaceutically acceptable acid/conjugate base with reasonable buffering capacity in the pH range acceptable for intravenous administration of the drug can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A preferred pH range for intravenous formulation wherein the drug has a hydroxamic acid moiety (e.g., as in PXD-101), can be about 9 to about 12. Consideration should be given to the solubility and chemical compatibility of the drug in choosing an appropriate excipient.

Subcutaneous formulations, preferably prepared according to procedures well known in the art at a pH in the range between about 5 and about 12, also include suitable buffers and isotonicity agents. They can be formulated to deliver a daily dose of drug in one or more daily subcutaneous administrations, e.g., one, two or three times each day. The choice of appropriate buffer and pH of a formulation, depending on solubility of the drug to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A preferred pH range for subcutaneous formulation wherein the drug has a hydroxamic acid moiety is about 9 to about 12. Consideration should be given to the solubility and chemical compatibility of the drug in choosing an appropriate excipient.

The drug can also be administered in intranasal form via topical use of suitable intranasal vehicles, or via transdermal routes, using those forms of transdermal skin patches well known to those of ordinary skill in that art. To be administered in the form of a transdermal delivery system, the administration will likely be continuous rather than intermittent throughout the dosage regime.

The further chemotherapeutic agent (or agents, if more than one is employed) may be administered using conventional methods and protocols well known to those of skill in the art. For example, a typical dosage rate for 5-fluorouracil (5-FU) is 750-1000 mg/m2 in a 24 hour period, administered for 4-5 days every 3 weeks. A typical dose rate for capecitabine is 1000 to 1250 mg/m2 orally, when administered twice daily on days 1 to 14 of every 3rd week.

In another embodiment of the disclosure, an article of manufacture containing materials useful for the treatment of the diseases or disorders described above is provided. The article of manufacture may comprise a container and a label or package insert on or associated with the container. Suitable containers include, for example, bottles, vials or syringes. The containers may be formed from a variety of materials such as glass or plastic. The container holds a composition that may be effective for treating the condition and may have a sterile access port (e.g., the container may be an intravenous solution bag or a vial having a stopper pierceable by a hypodermic injection needle). At least two active agents in the composition may be one or more methyltransferase inhibitors, such as methotrexate and one or more tyrosine kinase inhibitors. The label or package insert may indicate that the composition may be used for treating the condition of choice, such as cancer.

Moreover, the article of manufacture may comprise (a) a first container with a composition contained therein, wherein the composition comprises one or more methyltransferase inhibitors, such as methotrexate, and (b) a second container with a composition contained therein, wherein the composition comprises one or more receptor tyrosine kinase inhibitors. The article of manufacture in this embodiment of the disclosure may further comprise a package insert indicating that the first and second compositions can be used in combination to treat a disease or disorder including, for example, cancer. Additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

### Example 1

### Sensitivity of Cells to Treatment with an Antifolate

Cells with a different status of k-Ras (K-Ras mutant or k-Ras wild type) may be tested for their sensitivity to a drug such as an antifolate including, for example, a DHFR inhibitor.

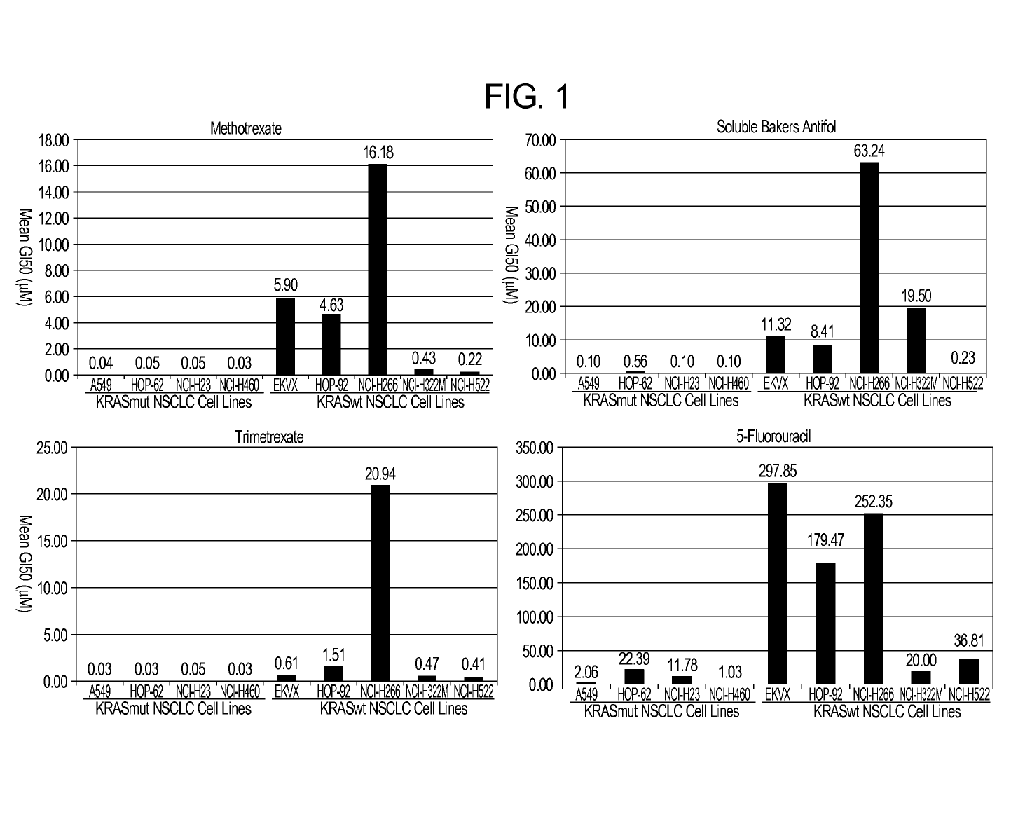

In an exemplary method, the NCI Developmental Therapeutics Program cancer drug screen database was also interrogated for association between K-RAS mutation status and drug efficacy in NCI60 NSCLC cell lines. This database compiles results from multiple experiments in which the NCI-60 bank of cell lines were treated with 5 doses of each drug and assayed for proliferation 48 hours later. Analysis of this data demonstrates lower G150 values for antifolates in K-RAS mutant versus K-RAS wild-type NSCLC cell lines. As such, this database revealed increased efficacy of antifolates in K-RAS mutant versus K-RAS wild-type NCI-60 NSCLC cell lines (see, FIG. 1). Additionally, a similar specificity was revealed for other anti-folate therapies in the NCI cell screen.

Additionally, a variety of NSCLC cell lines that were K-RAS mutant (A549, NCI-H460 & NCI-H23), K-RAS mutant/amplified (NCI-H727 & NCI-H2009) and K-RAS wild-type (Calu-3, NCI-H650 & NC-H661) NSCLC cells were plated in 96 well plates treated and treated 24 hours later with multiple concentrations of Methotrexate (0-10 μM). After an additional 72 hours cells were assayed for proliferation using the Invitrogen Cyquant Direct™ proliferation assay. IC50 (inhibitory concentration that kills 50% of cells) was determined using graphpad software. Cells were treated in triplicate and cell numbers were calculated as percent untreated control. K-RAS mutant (A549, NCI-H460 & NCI-H23) and K-RAS mutant/amplified (NCI-H727 & NCI-H2009) cells were sensitive to Methotrexate while K-RAS wild-type (Calu-3, NCI-H650 & NC-H661) cells were not sensitive to Methotrexate (see, FIG. 2).

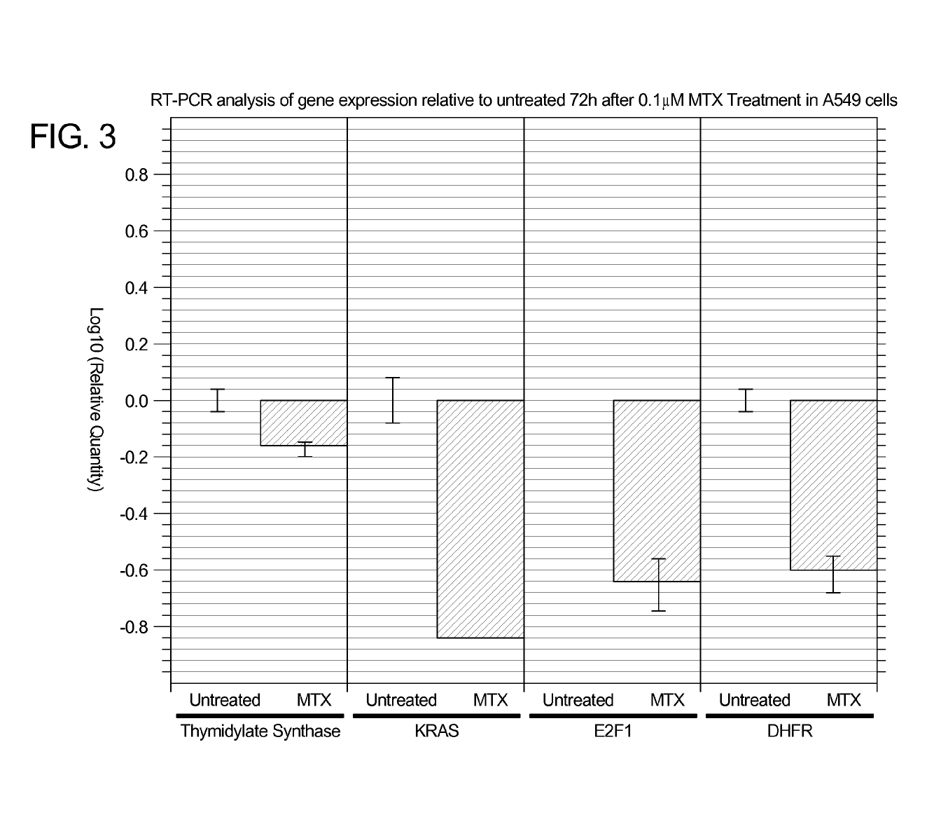

Finally, expression of genes/proteins related to folate metabolism and cell cycle progression were examined in K-RAS mutant and K-RAS wild-type NSCLC cells with Methotrexate treatment and K-RAS overexpression or knockdown. Briefly, A549 cells were treated with Methotrexate at a concentration of 0.1 μM. 72 hours after treatment, total RNA was extracted from treated and untreated cells. RT-PCR was then performed on extracted RNA to determine gene expression of K-RAS, Dihydrofolate Reductase (DHFR), Thymidylate Synthase (TYMS) and E2F1. Gene expression was normalized to Beta-2-Macroglobulin as an internal control. Expression of DHFR, TS, E2F-1, phosphorylated Rb and mutant K-RAS were decreased by Methotrexate treatment in K-RAS mutant but not in K-RAS wild-type cells (see, FIG. 3). Additionally, expression of DHFR, TS, E2F-1 and phosphorylated Rb are increased upon K-RAS transfection and decreased upon siRNA knockdown of mutant K-RAS. Examination of microarray gene expression data from the NCI-60 NSCLC cell lines demonstrates increased expression of folate metabolism associated genes in K-RAS mutant versus K-RAS wild-type cells.

Collectively, these studies highlight increased sensitivity to an antifolate in K-RAS mutant NSCLC cells. Without being bound to a theory of the invention, it is believed that mutant K-RAS drives expression and release of E2F-1 which may in turn lead to increased expression of DHFR/TS and potential dependency on these pathways.

### Example 2

### Responsiveness of K-Ras Mutant Tumors to Methotrexate Treatment In Vivo

Tumors with a different status of k-Ras (K-Ras mutant or k-Ras wild type) may be tested in vivo for their sensitivity to a drug.

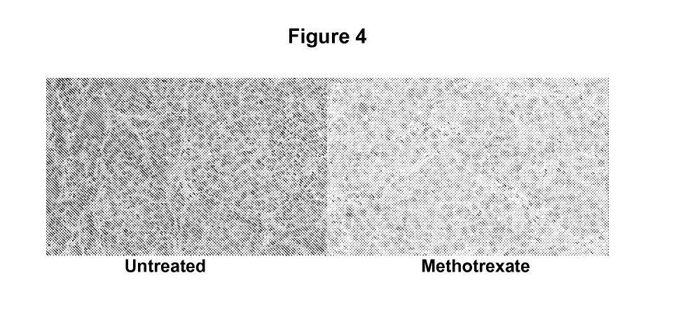

In an exemplary method, H460 cells determined to be sensitive to Methotrexate in Example 1 were implanted in mice and grown to approximately 500 mg before treatment with 130 mg/kg Methotrexate Q4Dx3 IP. Tumors were then harvested 10 days after treatment, fixed in formalin and stained for cleaved caspase-3. Next, bright-field pictures were taken at 40× (see, FIG. 4). Tumors with K-Ras mutant cells were shown to be sensitive (e.g., responsive) to Methotrexate.

### Example 3

### Determining Responsiveness of a Mammalian Subject to an Antifolate

The success of therapeutics in medicine and especially in a complex disease such as cancer depends on the correct diagnosis choice of patients treated with a drug. This process requires knowledge of the specific patient markers that can be used to predict how the patient will respond to a given drug or class of drugs that share a common mechanism of action. The inventors of the instant application have shown that cells which harbor a Ras mutation are responsive to an antifolate such as a DHFR inhibitor. A mammalian tumor likely to be responsive to a DHFR inhibitor may be identified as follows.

In an exemplary method, a biological sample was removed from subjects prior to treatment with an antifolate such as Methotrexate and analyzed for expression of one or more Ras mutations (e.g., one or more mutations in k-Ras (SEQ ID NO: 1), n-Ras (SEQ ID NO: 2) or h-Ras (SEQ ID NO: 3). The patient sample consisted of a tumor biopsy. The biological sample was then analyzed for the presence or absence of one or more Ras mutations (e.g., K-Ras mutations) and optionally one or more Ras amplifications. Patient samples which exhibited a Ras mutation (e.g., expression of mutated K-Ras) were determined to be responsive to treatment with the antifolate. Conversely, patient samples which did not exhibit a Ras mutation (e.g., wild-type K-Ras) were determined to not be responsive to treatment with antifolate.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method for predicting sensitivity of a test cell to a drug, the method comprising:
a. obtaining a test cell;
b. assaying the test cell for one or more Ras mutations;
c. assaying the test cell for amplification of a Ras gene;
d. determining if one or more Ras mutations are present or absent in the test cell and determining if an amplification of the Ras gene is present or absent in the test cell; and
e. employing the determination of the presence or absence of a Ras mutation in the test cell and the presence or absence of an amplification of Ras in the test cell to predict sensitivity of the test cell to the drug.

2. The method of claim 1, wherein Ras is k-Ras (SEQ ID NO: 1), n-Ras (SEQ ID NO: 2) or h-Ras (SEQ ID NO: 3).

3. The method of claim 2, wherein the k-Ras mutations are at one or more of positions 12, 13 or 61.

4. The method of claim 3, wherein the k-Ras mutations are selected from the group consisting of: G12A, G12N, G12R, G12C, G12S, G12V, G13N and Q61H.

5. The method of claim 2, wherein the h-Ras or n-Ras mutations are at one or more of positions 12, 13 or 61.

6. The method of claim 1, wherein the drug is a chemotherapeutic agent.

7. The method of claim 1, wherein the drug is an antifolate.

8. The method of claim 7, wherein the antifolate is a dihydrofolate reductase (DHFR) inhibitor.

9. The method of claim 8, wherein the DHFR inhibitor is Methotrexate or Pemetrexed.

10. The method of claim 1, wherein the drug is a tyrosine kinase inhibitor that targets HER1 (EGFR), HER2/neu, HER3, or any combination thereof.

11. The method of claim 10, wherein the tyrosine kinase inhibitor is an antibody.

12. The method of claim 11, wherein the antibody is monoclonal antibody.

13. The method of claim 12, wherein the monoclonal antibody is cetuximab (Erbitux), panitumumab, zalutumumab, nimotuzumab or matuzmab.

14. The method of claim 10, wherein the tyrosine kinase inhibitor is a small molecule inhibitor.

15. The method of claim 14, wherein the small molecule inhibitor is gefitinib, erlotinib or lapatinib.

16. The method of claim 1, wherein the test cell is obtained from a subject that has a disease or disorder.

17. The method of claim 16, wherein the disease or disorder is cancer.

18. The method of claim 17, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital urinary cancer and bladder cancer.

19. The method of claim 16, wherein the subject is a cancer patient.

20. The method of claim 1, wherein the test cell is assayed for one or more Ras mutations and an amplification of Ras by analyzing nucleic acid obtained from the test cell.

21. The method of claim 1, wherein the test cell is assayed for one or more Ras mutations by analyzing proteins obtained from the test cell.

22. The method of claim 1, wherein test cell is obtained from a tumor biopsy.

23. The method of claim 1, wherein the test cell is obtained from an aspirate, blood or serum.

24. The method of claim 1, wherein the test cell is predicted to be sensitive to the drug where one or more Ras mutations are determined to be present in the test cell and an amplification of Ras is determined to be present in the test cell.

25. The method of claim 1, wherein the test cell is predicted to be sensitive to the drug where one or more Ras mutations are determined to be present in the test cell and amplification of Ras is determined to be absent in the test cell.

26. The method of claim 1, wherein the test cell is predicted to be sensitive to the drug where Ras mutations are determined to be absent in the test cell and an amplification of Ras is determined to be present in the test cell.

27. The method of claim 1, wherein the test cell is predicted to be sensitive to the Drug where Ras mutations are determined to be absent in the test cell and amplification of Ras is determined to be absent in the test cell.

28. The method of claim 1, wherein the test cell is predicted to be insensitive to the drug where one ore more Ras mutations are determined to be present in the test cell and an amplification of Ras is determined to be present in the test cell.

29. The method of claim 1, wherein the test cell is predicted to be insensitive to the drug where one or more Ras mutations are determined to be present in the test cell and amplification of Ras is determined to be present in the test cell.

30. The method of claim 1, wherein the test cell is predicted to be insensitive to the drug where Ras mutations are determined to be absent in the test cell and an amplification of Ras is determined to be present in the test cell.

31. The method of claim 1, wherein the test cell is predicted to be insensitive to the drug where Ras mutations are determined to be absent in the test cell and amplification of Ras is determined to be absent in the test cell.

32. The method of claim 1, wherein the step of assaying the test cell for one or more Ras mutations and amplification of Ras is performed by in situ hybridization (TSH), northern blot, qRT-PCT or microarray analysis.


SEQUENCE LISTING (3 sequences)

SEQ ID NO: 1 - protein, 188 aa, human
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 2 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 3 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS